(19) United States
(12) Patent Application Publication
(10) Pub. No.: US 2014/0275234 A1
(43) Pub. Date: Sep. 18, 2014

(54) COMPOSITIONS AND METHODS OF USING CRYSTALLINE FORMS OF WORTMANNIN ANALOGS

(71) Applicants: Jeffrey Millard, Seattle, WA (US); Gary Christianson, Issaquah, WA (US); Eric Charles, Bothell, WA (US)

(72) Inventors: Jeffrey Millard, Seattle, WA (US); Gary Christianson, Issaquah, WA (US); Eric Charles, Bothell, WA (US)

(73) Assignee: ONCOTHYREON INC., Seattle, WA (US)

(21) Appl. No.: 14/233,325

(22) PCT Filed: Dec. 27, 2011
(86) PCT No.: PCT/US2011/067411

§ 371 Date: May 7, 2014

Related U.S. Application Data
(60) Provisional application No. 61/428,439, filed on Dec. 30, 2010.

(51) Int. Cl. C07D 311/78; A61K 45/06; A61K 31/366

(52) U.S. Cl. CPC C07D 311/78; A61K 31/366; A61K 45/06
USPC 514/453; 549/276

(57) ABSTRACT

Provided herein are novel crystalline forms of Compound 1. Also provided herein are compositions and methods of uses for the crystalline forms of Compound 1.

### CROSS REFERENCE

This application claims the benefit of U.S. Provisional Application No. 61/428,439 filed Dec. 30, 2010, which is incorporated by reference herein in its entirety.

### FIELD OF THE INVENTION

Described herein are certain crystalline forms of Compound 1 (chemical name, (4S,4aR,5R,6aS,9aR, Z)-1-((diallylamino)methylene)-11-hydroxy-4-(methoxymethyl)-4-a,6a-dimethyl-2,7,10-trioxo-1,2,4,4a,5,6,6a,7,8,9,9a,10-dodecahydroindeno[4,5-h]isochromen-5-yl acetate, also known with the chemical name acetic acid 4-diallylaminomethylene-6-hydroxy-1-α-methoxymethyl-10β,13β-dimethyl-3,7,17-trioxo-1,3,4,7,10,11β,12,13,14α,15,16,17-dodecahydro-2-oxa-cyclopenta[a]phenanthren-11-yl ester, also known as PX-866, a small molecule drug that irreversibly inhibits phosphatidylinositol-3-kinase (PI-3K).

[chemical structure]

### BACKGROUND OF THE INVENTION

The PI-3kinases (PI-3Ks) are a family of related intracellular signal transducer enzymes capable of phosphorylating the 3 position hydroxyl group of the inositol ring of phosphatidylinositol (PtdIns). Key nodes of the PI-3K intracellular signaling pathway are frequently mutated in cancer. In normal cells the PI-3K pathway is tightly controlled. Inappropriate activation or mutation of PI-3K is important in the pathogenesis of many human cancers.

### SUMMARY OF THE INVENTION

Polymorphism of a compound is often of significant importance in pharmacy and pharmacology. Polymorphs, crystals of the same molecule, often have different physical properties as a result of the order of the molecules in the crystal lattice. The differences in physical properties of polymorphic forms affect pharmaceutical parameters such as storage stability, compressibility and density (important in formulation and product manufacturing), and dissolution rates (important in determining bioavailability).

Amorphous PX-866 has been previously used for clinical studies. The present disclosure provides certain novel forms of PX-866 which remove some disadvantages associated with amorphous PX-866.

Provided herein are novel crystalline forms of PX-866 (Compound 1). Also provided herein are compositions and methods of uses thereof for the crystalline forms of Compound 1. In some embodiments, the novel crystalline forms of Compound 1 present better physical properties than amorphous form.

Provided herein are crystalline forms of a compound having a structural formula

[chemical structure]

which are substantially free of wortmannin.

In one aspect, provided herein is a crystalline form of a compound having a structural formula

[chemical structure]

wherein the form is
(a) a crystalline anisole solvate; and
(b) has an X-ray powder diffraction pattern (XRPD) with characteristic peaks at 7.9±0.1 degrees 2-Theta, 8.5±0.1 degrees 2-Theta, 10.2±0.1 degrees 2-Theta, 11.1±0.1 degrees 2-Theta, 14.0±0.1 degrees 2-Theta, 14.2±0.1 degrees 2-Theta, 17.9±0.1 degrees 2-Theta, 18.7±0.1 degrees 2-Theta, 21.0±0.1 degrees 2-Theta, 21.2±0.1 degrees 2-Theta, and 28.2±0.1 degrees 2-Theta.

Provided herein is a crystalline anisole solvate form of PX-866 having an XRPD of FIG. 1.

In one aspect, provided herein is a crystalline form of a compound having a structural formula

[chemical structure]

wherein the form is
(a) a crystalline anisole solvate; and
(b) has an X-ray powder diffraction pattern (XRPD) with characteristic peaks at 10.2±0.1 degrees 2-Theta, 11.1±0.1 degrees 2-Theta, 14.0±0.1 degrees 2-Theta, 14.2±0.1 degrees 2-Theta, and 21.0±0.1 degrees 2-Theta.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

The crystalline form described above exhibits a predominant endotherm at about 146° C. as measured by Differential Scanning calorimeter. In some of such embodiments, the endotherm is observed when using a scan rate of 10° C. per minute.

In one embodiment, provided herein are crystalline forms of Compound I, wherein the forms have a general space group P2₁2₁2₁.

The crystalline form described above exhibits a single crystal X-ray crystallographic analysis at 120 K with the following crystal parameters:

| Space Group | P2₁2₁2₁ |
|---|---|
| a, Å | 13.7140(3)° |
| b, Å | 15.4272(4) |
| c, Å | 15.6890(4) |
| α | 90 |
| β | 90 |
| γ | 90 |
| Z (molecules/unit cell) | 4° |
| Calculated Density (g/cm) | 1.268.° |

Provided herein is a crystalline form of a compound having a structural formula

[chemical structure]

wherein the crystalline form exhibits a predominant endotherm at about 146° C. as measured by Differential Scanning calorimeter.

Provided herein is a method of preparing a crystalline solvate form of a compound having a structural formula

[chemical structure]

that is substantially free of wortmannin, comprising cooling down a supernatant, solution, suspension, dispersion or emulsion of the compound in a suitable solvent to a temperature of between 4° C. to −20° C.

In some embodiments of the method described above, the supernatant, solution, suspension, dispersion or emulsion comprises a solvent selected from toluene, anisole, cumene, propyl acetate, 4-methyl-2-pentanone, chlorobenzene, or 1-pentanol, or a combination thereof.

In some embodiments, the supernatant, solution, suspension, dispersion or emulsion comprises anisole as described in Example 5.

Provided herein are methods of preparing a crystalline solvate form of a compound having a structural formula

[chemical structure]

comprising adding an antisolvent to a supernatant, solution, suspension, dispersion or emulsion of the compound in a solvent, wherein Compound 1 has differential solubility in the solvent compared to the antisolvent.

In some embodiments, the methods comprise optionally cooling the supernatant, solution, suspension, dispersion or emulsion of the compound in a solvent to a temperature of between 4° C. to −20° C. prior to adding an antisolvent.

In some embodiments of the methods, the solvent is selected from tetrahydrofuran (THF), water, acetonitrile, acetone, n-butanol, sec-butanol, butyl acetate, tert-butylmethyl ether (TBME), chloroform, 1,2-dichloroethane, N,N-dimethylacetamide, N,N-dimethylformamide, dimethyl sulfoxide, ethanol, 1,4-dioxane, ethyl acetate, isopropyl acetate, isobutyl acetate, 2-ethoxyethanol, ethylene glycol, formamide, methanol, 2-methoxyethanol, methylbutyl ketone, N-methylpyrrolidone, nitromethane, pyridine, sulfolane, toluene, xylene, anisole, hexane, cyclohexane, methylcyclohexane, cumene, propyl acetate, chlorobenzene, pentane, 1-pentanol, 4-methyl-2-pentanone, and 1,1,2-trichloroethene, or a combination thereof.

In some embodiments of the methods, the antisolvent is selected from water, toluene, anisole, cumene, propyl acetate, 4-methyl-2-pentanone, chlorobenzene, or 1-pentanol, or a combination thereof.

In some embodiments of the methods, the solvent is selected from THF, acetonitrile, MTBE, ethylene glycol, acetone, ethyl acetate, and ethanol, and the antisolvent is selected from toluene, anisole, cumene, propyl acetate, 4-methyl-2-pentanone, chlorobenzene, or 1-pentanol.

In some embodiments, the solvent is anisole and the antisolvent is hexane, heptanes, cyclohexane or water.

Provided herein are methods of preparing a crystalline PX-866 form having a XRPD of FIG. 1 comprising

(a) dissolving Compound 1 in tetrahydrofuran (THF)
(b) adding anisole to the mixture of step (a); and
(c) concentrating the mixture of step (b).

In some embodiments of the method described above, the solution of step (a) is optionally cooled to a temperature of between about −4° C. to about 15° C., preferably between about 0° C. to about 10° C. prior to step (b). In some other embodiments of the method described above, the solution of step (a) is optionally held at a temperature higher than ambient temperature (e.g., between about 25° C. to about 100° C., preferably between about 25° C. to about 50° C.) prior to step (b).

In some embodiments of the method described above, the solution of step (b) is optionally seeded with crystals of claim 2, prior to step (c).

In some embodiments of the method described above, the mixture of step (c) is optionally cooled, (e.g., maintained at a temperature of between about 0° C. to about 10° C.) for a period of time during concentration and then allowed to warm to ambient temperature while continuing the concentration.

In some of the embodiments described above, the mixture of step (b) is stirred for a period of time (e.g., between about 2-8 hours) at a temperature at or below ambient temperature (e.g., between about 0° C. to 10° C., between about 15° C. to about 25° C.). In some embodiments, the concentration is carried out, for example, via slow vacuum distillation at a temperature at or below ambient temperature (e.g., between about 0° C. to 10° C., between about 15° C. to about 25° C.) for a period of time (e.g., between about 2-8 hours) to obtain a slurry. Optionally the slurry is warmed to ambient temperature while continuing vacuum distillation, then the mixture is filtered and the crystalline product is dried.

In some embodiments of the method described above, the seed crystals are prepared by

(a) dissolving or suspending PX-866 in anisole at ambient temperature; and
(b) cooling the supernatant from step (a) to 4° C. and then to −20° C.

In some embodiments of the method described above, the resultant solid is optionally dried under nitrogen stream or under vacuum.

Provided herein is a pharmaceutically acceptable composition comprising PX-866, anisole and a pharmaceutically acceptable carrier.

In some embodiments, the pharmaceutical composition is formulated for intravenous injection, subcutaneous injection, sublingual administration, rectal administration, buccal administration, oral administration, topical administration, transdermal administration, or inhaled administration.

In some embodiments, the composition is a tablet.

Provided herein is a pharmaceutical composition comprising a crystalline anisole solvate form of Compound I, i.e., PX-866, and a pharmaceutically acceptable carrier.

In some embodiments, for any of the pharmaceutical compositions described above or below, Compound 1 is present in a unit dosage form in an amount of about 0.1 to 20 mg.

In some embodiments, for any of the pharmaceutical compositions described above or below, the pharmaceutical composition further comprises a second anti-cancer agent.

In some embodiments, for any of the pharmaceutical compositions described above or below, the composition further comprises one or more additional crystalline forms of Compound 1 selected from a solvate form, a co-crystal, or a solvate-hydrate.

In some embodiments, the one or more additional crystalline forms of Compound 1 are selected from a toluene solvate having an XRPD pattern of FIG. 4; a propyl acetate solvate having an XRPD pattern of FIG. 6; a 4-methyl-2-pentanone solvate form having an XRPD pattern of FIG. 9; a cumene solvate having an XRPD pattern of FIG. 10; a 1-pentanol solvate having an XRPD pattern of FIG. 11; and a chlorobenzene solvate form having an XRPD pattern of FIG. 12.

Provided herein are methods of treating cancer in a subject in need thereof comprising administering a composition comprising a therapeutically effective amount of any crystalline form described above to the subject in need thereof.

In some embodiments of the methods the cancer is selected from the group consisting of breast cancer, lung cancer, head and neck cancer, brain cancer, abdominal cancer, colon cancer, colorectal cancer, esophageal cancer, parapharyngeal cancer, gastrointestinal cancer, glioma, liver cancer, tongue cancer, neuroblastoma, osteosarcoma, ovarian cancer, renal cancer, pancreatic cancer, retinoblastoma, cervical cancer, uterine cancer, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer including castration resistant forms, ovarian cancer including mucinous ovarian carcinoma, squamous cell carcinoma of the head and neck, colorectal carcinoma, glioblastoma, cervical carcinoma, endometrial carcinoma, gastric carcinoma, pancreatic carcinoma, leiomyosarcoma, breast carcinoma, adenocystic carcinoma, neuroendocrine tumors, brain tumors, cancer of the central nervous system, glioblastoma, and blastomas.

In some embodiments of the methods, the cancer is squamous cell carcinoma of the head and neck, non-small cell lung cancer, colon cancer or prostate cancer.

In some embodiments, the method further comprises administering an anti-cancer agent.

Also provided herein is a method of treating a fibrotic condition in a subject in need thereof comprising administering a composition comprising a therapeutically effective amount of the crystalline anisole solvate form of Compound 1 to the subject in need thereof.

Also provided herein is a method of treating pulmonary fibrosis in a subject in need thereof comprising administering a composition comprising a therapeutically effective amount of the crystalline anisole solvate form of Compound 1 to the subject in need thereof.

In a further aspect, provided herein is PX-866 (including amorphous or crystalline PX-866) that is substantially free of wortmannin. In some embodiments, PX-866 that is substantially free of wortmannin comprises less than 0.2% wortmannin. In some embodiments, PX-866 that is substantially free of wortmannin comprises less than 0.15% wortmannin. In some embodiments, PX-866 that is substantially free of wortmannin comprises less than 0.1% wortmannin. In some embodiments, PX-866 that is substantially free of wortmannin comprises less than 0.05% wortmannin. In some embodiments, PX-866 that is substantially free of wortmannin comprises less than 0.01% wortmannin. In some embodiments, PX-866 that is substantially free of wortmannin comprises undetectable levels or wortmannin when tested (e.g., by High Pressure Liquid Chromatography (HPLC) or Gas Chromatography (GC)).

In one aspect, provided herein is a crystalline form of a compound having a structural formula

[chemical structure]

wherein the form is
(a) a crystalline propyl acetate solvate; and
(2) has an X-ray powder diffraction pattern with at least two characteristic peaks having 2-theta values selected from 8.0±0.1 degrees 2-Theta, 8.4±0.1 degrees 2-Theta, 10.2±0.1 degrees 2-Theta, 11.0±0.1 degrees 2-Theta, 14.0±0.1 degrees 2-Theta and 19.2±0.1 degrees 2-Theta.

Provided herein is a crystalline propyl acetate solvate form of PX-866 having an XRPD of FIG. 6.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

The crystalline form described above exhibits a predominant endotherm at about 80° C. as measured by Differential Scanning calorimeter. In one embodiment, the scan rate is 10° C. per minute.

The crystalline form described above exhibits a single crystal x-ray crystallographic analysis at 100 K with the following crystal parameters:

| Space Group | P2₁2₁2₁ |
|---|---|
| a, Å | 13.4963(5)° |
| b, Å | 15.5158(5) |
| c, Å | 15.6912(6) |
| α | 90 |
| β | 90 |
| γ | 90 |
| Z (molecules/unit cell) | 4° |
| Calculated Density (g/cm) | 1.269.° |

Provided herein is a crystalline form of a compound having a structural formula

[chemical structure]

wherein the crystalline form exhibits a predominant endotherm at about 80° C. as measured by Differential Scanning calorimeter.

In another aspect, provided herein is a crystalline form of a compound having a structural formula

[chemical structure]

wherein the form is
(a) a crystalline toluene solvate; and
(2) has an X-ray powder diffraction pattern with at least two characteristic peaks having 2-theta values selected from 12.5±0.1 degrees 2-Theta, 14.0±0.1 degrees 2-Theta, and 21.1±0.1 degrees 2-Theta.

Provided herein is a crystalline toluene solvate form of PX-866 having an XRPD of FIG. 4.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

The crystalline form described above exhibits a predominant endotherm at about 142.0° C. as measured by Differential Scanning calorimeter. In one embodiment, the scan rate is 10° C. per minute.

In another aspect, provided herein is a crystalline form of a compound having a structural formula

[chemical structure]

wherein the form is
(a) a crystalline cumene solvate; and
(2) has an X-ray powder diffraction pattern with at least two characteristic peaks having 2-theta values selected from 7.8±0.1 degrees 2-Theta, 8.4±0.1 degrees 2-Theta, 10.1 degrees 2-Theta±0.1 degrees 2-Theta, 10.7±0.1 degrees 2-Theta, 13.7±0.1 degrees 2-Theta, 14.1±0.1 degrees 2-Theta, 18.1±0.1 degrees 2-Theta, 18.9±0.1 degrees 2-Theta, 20.6±0.1 degrees 2-Theta, and 20.8±0.1 degrees 2-Theta.

Provided herein is a crystalline cumene solvate form of PX-866 having an XRPD of FIG. 10.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

In another aspect, provided herein is a substantially pure crystalline form of a compound having a structural formula

[chemical structure]

wherein the form is
(a) a crystalline 4-methyl-2-pentanone solvate; and
(2) has an X-ray powder diffraction pattern with at least two characteristic peaks having 2-theta values selected from 7.9, ±0.1 degrees 2-Theta, 8.4, ±0.1 degrees 2-Theta, 10.2, ±0.1 degrees 2-Theta, 10.9, ±0.1 degrees 2-Theta, 13.9, ±0.1 degrees 2-Theta, 14.2, ±0.1 degrees 2-Theta, 18.5, ±0.1 degrees 2-Theta, 19.2, ±0.1 degrees 2-Theta, and 20.7±0.1 degrees 2-Theta.

Provided herein is a crystalline 4-methyl-2-pentanone solvate form of PX-866 having an XRPD of FIG. 9.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

In a further aspect, provided herein is a substantially pure crystalline form of a compound having a structural formula

[chemical structure]

wherein the form is
(a) a crystalline 1-pentanol solvate; and
(2) has an X-ray powder diffraction pattern with at least two characteristic peaks having 2-theta values selected from 8.1±0.1 degrees 2-Theta, 8.5±0.1 degrees 2-Theta, 10.2±0.1 degrees 2-Theta, 11.1±0.1 degrees 2-Theta, 12.5±0.1 degrees 2-Theta, 14.0±0.1 degrees 2-Theta, 14.3±0.1 degrees 2-Theta, 17.9±0.1 degrees 2-Theta, 18.8±0.1 degrees 2-Theta, 20.7±0.1 degrees 2-Theta, and 21.3±0.1 degrees 2-Theta.

Provided herein is a crystalline 1-pentanol solvate form of PX-866 having an XRPD of FIG. 11.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

In yet another aspect, provided herein is a substantially pure crystalline form of a compound having a structural formula

[chemical structure]

wherein the form is
(a) a crystalline chlorobenzene solvate; and
(2) has an X-ray powder diffraction pattern with at least two characteristic peaks having 2-theta values selected from 8.0±0.1 degrees 2-Theta, 8.5±0.1 degrees 2-Theta, 10.3±0.1 degrees 2-Theta, 11.1±0.1 degrees 2-Theta, 14.1±0.1 degrees 2-Theta, 17.9±0.1 degrees 2-Theta, 18.8±0.1 degrees 2-Theta, 19.1±0.1 degrees 2-Theta, 21.0±0.1 degrees 2-Theta and 28.3±0.1 degrees 2-Theta.

Provided herein is a crystalline chlorobenzene solvate form of PX-866 having an XRPD of FIG. 12.

In some embodiments, the crystalline form described above is a substantially pure crystalline form. In some embodiments, the crystalline form described above has a purity of at least 90%. In some embodiments, the crystalline form described above has a purity of at least 95%. In some embodiments, the crystalline form described above has a purity of at least 98%.

For any crystalline forms described above or below, the crystalline forms exhibit higher stability at ambient temperature than amorphous form.

In some embodiments, a crystalline solvate form of Compound 1 and/or analogs thereof has a form with the general space group P2₁2₁2₁.

In other embodiments, crystalline forms of Compound 1 and/or analogs thereof allow for ease of formulation compared to the amorphous form of Compound 1. In some instances, crystalline forms of Compound 1 and/or analogs thereof have better flowability as compared to the amorphous form.

In any of the embodiments described above, the pharmaceutical composition comprising a crystalline solvate form of Compound 1 further comprises a second anti-cancer agent.

In any of the embodiments described above, the pharmaceutical composition comprising a crystalline anisole solvate form of Compound 1 further comprises one or more additional crystalline forms of Compound 1 selected from a solvate form, a co-crystal, or a solvate-hydrate.

In any of the embodiments described above or below, any crystalline form of PX-866 (including any crystalline solvate form of PX-866, any crystalline solvate-co-crystal form of PX-866, and/or any solvate-hydrate form of PX-866) is optionally recrystallized a second time (or more times) from a suitable solvent or mixture of solvent and antisolvent.

In some embodiments provided herein are pharmaceutical compositions comprising or derived from the crystalline forms and a pharmaceutically acceptable carrier. In some embodiments, the crystalline form of Compound 1 in the pharmaceutical compositions is present in a unit dosage form at an amount of between about 0.1 to about 20 mg. In certain embodiments, the pharmaceutical compositions are formulated for intravenous injection, subcutaneous injection, sublingual administration, rectal administration, buccal administration, oral administration, topical administration, transdermal administration, or inhaled administration. In certain embodiments, the pharmaceutical composition is a tablet, a pill, a capsule, a suspension, a gel, a dispersion, a solution, an emulsion, a micronized powder, a lozenge, a transdermal patch, an ointment, or a lotion. In certain embodiments, the pharmaceutical composition is a tablet. In some embodiments, the tablet is formulated for immediate release, delayed release, controlled release, or combinations thereof. In certain embodiments, the pharmaceutical composition further comprises a second therapeutically active agent. In certain embodiments, the pharmaceutical composition further comprises a second anti-cancer agent.

In some embodiments, the present invention also provides methods of treating cancer comprising administering to a subject in need thereof any crystalline form of Compound 1 and/or analog thereof described above or below, or a pharmaceutical composition comprising the crystalline form.

Cancers treatable by methods described herein include, but are not limited to, breast cancer, lung cancer, head and neck cancer, brain cancer, abdominal cancer, colon cancer, colorectal cancer, esophageal cancer, parapharyngeal cancer, gastrointestinal cancer, glioma, liver cancer, tongue cancer, neuroblastoma, osteosarcoma, ovarian cancer, renal cancer, pancreatic cancer, retinoblastoma, cervical cancer, uterine cancer, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer including castration resistant forms, ovarian cancer including mucinous ovarian carcinoma, and/or carcinomas including but not limited to squamous cell carcinoma of the head and neck, colorectal carcinoma, glioblastoma, cervical carcinoma, endometrial carcinoma, gastric carcinoma, pancreatic carcinoma, leiomyosarcoma, breast carcinoma, adenocystic carcinoma, neuroendocrine tumors, including pancreatic neuroendocrine tumors, brain tumors, cancers of the central nervous system, glioblastoma, and blastomas. In certain embodiments, the cancer is head and neck cancer, lung cancer, colon cancer, breast cancer, ovarian cancer, or prostate cancer.

In some embodiments, the methods of treatment described herein treat a lung cancer such as non-small cell lung cancer (NSCLC). In other embodiments, the methods of treatment described herein treat a head and neck cancer such as squamous cell carcinoma of the head and neck (SCCHN). In some embodiments, the methods of treatment described herein treat a carcinoma such as a mucinous ovarian carcinoma or a colorectal carcinoma. In some embodiments, the methods of treatment described herein treat a pancreatic cancer such as a pancreatic neuroendocrine tumor.

In some embodiments, the also provided are methods of treating a fibrosis comprising administering to a subject in need thereof any crystalline form of Compound 1 described above and/or analog thereof and/or a pharmaceutical composition comprising the crystalline form.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the embodiments described herein are set forth with particularity in the appended claims. A better understanding of the features and advantages of the present embodiments will be obtained by reference to the following detailed description that sets forth illustrative embodiments, in which the principles of the embodiments are utilized, and the accompanying drawings of which:

FIG. 1 shows the X-ray powder diffraction pattern of an anisole solvate of PX-866; redrawn by draftsman.

FIG. 2 shows the DSC thermogram of an anisole solvate of PX-866; redrawn by draftsman.

FIG. 3 shows the a-axis projection of the crystal packing in an anisole solvate of PX-866.

FIG. 4 shows the X-ray powder diffraction pattern of a toluene solvate of PX-866; redrawn by draftsman.

FIG. 5 shows the DSC thermogram of a toluene solvate of PX-866; redrawn by draftsman.

FIG. 6 shows the X-ray powder diffraction pattern of a propyl acetate solvate of PX-866; redrawn by draftsman.

FIG. 7 shows the DSC thermogram of a propyl acetate solvate of PX-866; redrawn by draftsman.

FIG. 8 shows the a-axis projection of the crystal packing in a propyl acetate solvate of PX-866.

FIG. 9 shows the X-ray powder diffraction patterns of the crystalline PX-866 prepared from a solution comprising 4-methyl-2-pentanone; redrawn by draftsman.

FIG. 10 shows the X-ray powder diffraction pattern of the crystalline PX-866 prepared from a solution comprising cumene; redrawn by draftsman.

FIG. 11 shows the X-ray powder diffraction pattern of the crystalline PX-866 prepared from a solution comprising 1-pentanol; redrawn by draftsman.

FIG. 12 shows the X-ray powder diffraction pattern of the crystalline PX-866 prepared from a solution comprising chlorobenzene; redrawn by draftsman.

FIG. 13 shows results of stability testing at 40° C., 75% relative humidity between crystalline anisole solvate, toluene solvate, propyl acetate solvate and amorphous forms of PX-866; redrawn by draftsman.

FIG. 14 has two panels, Panel A and Panel B and shows in vitro dose response of PX-866 in platelets and tumor derived cell lines. Panel A). PX-866 was added to whole blood from three different donors and samples were incubated for 2 hours at 37° C. Platelets were isolated from the samples and stimulated with TRAP for 15 min. Sample lysates were subsequently analyzed for p-Akt and total-Akt levels by ELISA. Calculated IC50 values are shown for each donor sample; redrawn by draftsman. Panel B). Tumor cell lines were stimulated with IGF1 in the absence or presence of various amounts of PX-866 for 10 min. Sample lysates were subsequently analyzed for p-Akt and total-Akt levels by ELISA. Calculated IC50 values are shown for each cell line; redrawn by draftsman.

FIG. 15 shows mean plasma concentration profiles for 17-hydroxy PX-866 following administration of amorphous PX-866 and crystalline anisole solvate form of PX-866 in humans showing that the amorphous and crystalline forms have similar profiles; redrawn by draftsman.

FIG. 16(A) shows a micrograph of amorphous PX-866 at 10× magnification using non-polarized light.

FIG. 16(B) shows a micrograph of amorphous PX-866 at 10× magnification using polarized light.

FIG. 17(A) shows a micrograph of PX-866 anisole solvate at 10× magnification using non-polarized light.

FIG. 17(B) shows a micrograph of PX-866 anisole solvate at 10× magnification using polarized light.

### DETAILED DESCRIPTION OF THE INVENTION

Compound 1 (chemical name, (4S,4aR,5R,6aS,9aR,Z)-1-((diallylamino)methylene)-11-hydroxy-4-(methoxymethyl)-4-a,6a-dimethyl-2,7,10-trioxo-1,2,4,4a,5,6,6a,7,8,9,9a,10-dodecahydroindeno[4,5-h]isochromen-5-yl acetate, also known with the chemical name acetic acid 4-diallylaminomethylene-6-hydroxy-1-α-methoxymethyl-10β,13β-dimethyl-3,7,17-trioxo-1,3,4,7,10,11β,12,13,14α,15,16,17-dodecahydro-2-oxa-cyclopenta[a]phenanthren-11-yl ester, also known as PX-866) is a small molecule drug that irreversibly inhibits phosphatidylinositol-3-kinase. Compound 1 has the following structural formula:

[chemical structure]

### Identification of Non-Amorphous Forms of PX-866

Typically, Compound 1 is prepared from wortmannin in a number of synthetic steps. Compound 1 is prepared as an orange oil after chromatography. See, for example U.S. Pat. No. 7,081,475. Polymorphic forms of Compound 1 have not been identified to date. The polymorphs of the embodiments described herein encompass racemates, racemic mixtures, and diastereomeric mixtures with all possible isomers and mixtures thereof of Compound 1.

Because of the structural similarity between the starting material, wortmannin, and Compound 1, purification of Compound 1 (e.g., chromatography) presents a challenge. Accordingly, provided herein are certain conditions for purification and/or crystallization of Compound 1 and/or analogs thereof.

The amorphous form of PX-866 shows variable lot to lot residual solvent and purity levels and contains low levels of the starting material wortmannin. Further, amorphous PX-866 has poor stability at ambient temperature and tends to form a sticky solid which causes difficulties during manufacturing processes.

Accordingly, some desired objectives in the search for improved forms of PX-866 were crystallinity, removal of starting material in the final product, stability and ease of handling while retaining solubility and reproducibility between lots. Described herein are crystalline forms of PX-866 which are improved forms of PX-866.

In addition, amorphous PX-866 is manufactured as capsules by slow-filling of the unprocessed, unblended amorphous PX-866 into capsules by weight. The amorphous material is often sticky and tends to clump (e.g., form a sticky mass) which leads to difficulties during manufacturing. Tablet forms are not easy to manufacture with amorphous PX-866.

Further, the capsule product is only tested for disintegration, instead of dissolution, because of variability in the time required to allow complete solution access to the powder present in the capsule, and poor stability of PX-866 in solution. As the capsule hydrates, some powder is wetted earlier than the remaining powder in the capsule, and plugs can form which retard the release of a portion of the active agent PX-866 from the capsule.

Accordingly, some desired objectives in developing an improved dosage form were scalability and manufacturability, improved purity, ability to blind future studies, blend uniformity, flowability, and reduced moisture content. Tablets were desired over capsules to improve efficiency of manufacturing and avoid the drawbacks of the capsule formulation described above. Described herein are crystalline forms of PX-866 which achieve the desired objectives for developing improved dosage forms. Further, the crystalline forms described herein allow faster filtration, faster drying, and easier recovery from filters and reaction vessels thus allowing for ease of handling.

### Studies to Identify Non-Amorphous Forms of PX-866

An initial solvent screen was performed on PX-866 to determine appropriate primary solvents and anti-solvents. Slurries and several precipitations were then performed utilizing several primary solvents and anti-solvents at 50 mg scale. Following drying, the material isolated from the slurry in pure deionized water was shown to significantly reduce the heptane content. The water slurry was then scaled up to 0.5 g and performed at ambient temperature. Since there was a concern for degradation taking place during the slurry at ambient temperature an additional slurry was performed on 0.5 g scale at 10° C. in an attempt to minimize any potential degradation. Material isolated from the 10° C. slurry did not show a significant reduction in heptane content. The solids isolated from the ambient temperature water slurry afforded a heptane content of 3,800 ppm, which is below the technical requirements for registration of pharmaceuticals for human use limit (5,000 ppm); however a notable decrease in PX-866 purity was also observed.

Results from the initial solvent screen are shown in Table 1A and Table 2A. This initial solvent screen of PX-866 in 18 solvents was executed to select appropriate solvents for slurry experiments. Approximately 1-2 mg of the material was placed in 2 dram glass vials and the chosen solvents were added in 50 μL portions to determine the minimum amount of solvent needed for dissolution. The vials were shaken at ambient temperature and visually inspected between each addition to determine if complete dissolution had taken place. As shown in Table 1A, PX-866 displayed good solubility in the following solvents at ambient temperature and were designated as primary solvents: acetonitrile (MeCN), dioxane, acetone, MTBE, ethanol (EtOH), ethyl acetate (EtOAc), isopropyl acetate (IPAc), isopropyl alcohol (IPA), tetrahydrofuran (THF), methyl ethyl ketone (MEK), dimethylformamide (DMF), acetic acid (AcOH), methanol (MeOH), toluene and dichloromethane (DCM).

TABLE 1A
Initial Solvent Screen Results with Heptane as Anti-solvent

| | | | | | | | Appearance |
|---|---|---|---|---|---|---|---|
| | | | Vial | | | Amt of | after |
| | Material | Solvent | Conc. | Temp. | | Heptane | heptane |
| Solvent | Amt (mg) | Amt (mL) | (mg/mL) | (° C.) | Soluble | (mL) | addition |
| MeCN | 0.8 | 0.05 | 16.0 | RT | Yes | 0.2 | Oil |
| Dioxane | 1.9 | 0.05 | 38.0 | RT | Yes | 0.15 | Cloudy |
| Acetone | 1.9 | 0.05 | 38.0 | RT | Yes | 0.15 | Cloudy |
| MTBE | 1.3 | 0.10 | 13.0 | RT | Yes | 0.05 | Precipitate |
| EtOH | 2.8 | 0.05 | 56.0 | RT | Yes | 0.2 | Cloudy |
| EtOAc | 1.9 | 0.05 | 38.0 | RT | Yes | 0.15 | Precipitate |
| IPAc | 1.6 | 0.05 | 32.0 | RT | Yes | 0.1 | Precipitate |
| IPA | 1.4 | 0.05 | 28.0 | RT | Yes | 0.2 | Cloudy |
| THF | 2.6 | 0.05 | 52.0 | RT | Yes | 0.15 | Precipitate |
| MEK | 2.8 | 0.05 | 56.0 | RT | Yes | 0.15 | Cloudy |
| DMF | 2.8 | 0.05 | 56.0 | RT | Yes | 0.2 | Two layers |
| AcOH | 2.4 | 0.05 | 48.0 | RT | Yes | 0.15 | Oil |
| MeOH | 2.6 | 0.05 | 52.0 | RT | Yes | 0.15 | Two layers |
| c-Hexane | 2.1 | 7.00 | 0.3 | RT | No | n/a | n/a |
| Heptane | 2.8 | 7.50 | 0.4 | RT | No | n/a | n/a |
| DCM | 2.4 | 0.05 | 48.0 | RT | Yes | 0.15 | Cloudy |
| Toluene | 2.8 | 0.05 | 56.0 | RT | Yes | 0.05 | Precipitate |
| Water | 2.5 | 7.50 | 0.3 | RT | Partially | n/a | n/a |

TABLE 2A
Initial Solvent Screen Results with H₂O as Anti-solvent

| | | | Vial | | | Amt of | Appearance |
|---|---|---|---|---|---|---|---|
| | Material | Solvent | Conc. | Temp. | | H₂O | after H₂O |
| Solvent | Amt (mg) | Amt (mL) | (mg/mL) | (° C.) | Soluble | (mL) | addition |
| Acetone | 5.1 | 0.05 | 102.0 | RT | Yes | 0.1 | Precipitate |
| EtOH | 8.2 | 0.05 | 164.0 | RT | Yes | 0.1 | Precipitate |
| IPA | 10.6 | 0.10 | 106.0 | RT | Yes | 0.25 | Precipitate |
| MeOH | 7.5 | 0.05 | 150.0 | RT | Yes | 0.05 | Precipitate |
| MECN | 5.0 | 0.05 | 100.0 | RT | Yes | 0.15 | Cloudy |

PX-866 showed limited or no solubility in c-hexane, heptane, and water and these solvents were thus designated as anti-solvents. To estimate the ratio of primary solvent to anti-solvent necessary for use in the slurry experiments, heptane or water was then added to the dissolved PX-866 in 50 mL, portions until a turbid solution was formed or solids precipitated out. As shown in Tables 1A and 2A, precipitation or turbidity was observed in most experiments where the anti-solvent was miscible with the primary solvent. The exceptions occurred when MeCN and AcOH were used as the primary solvent as the product oiled out upon anti-solvent addition.

Further slurry experiments were performed using approximately 50 mg of PX-866 and 0.5-1.0 mL of premixed solvent systems. The solvent systems were chosen based on the results from the initial solvent screen. The PX-866 was weighed into vials where the desired solvent system was added. The resulting mixtures were then allowed to stir with magnetic stirring at ambient temperature. Only three solvent systems [THF/heptane (1:10), MTBE/heptane (2:1) and toluene/heptane (1:1)] afforded slurries with free flowing solids, as shown in Table 3. Two solvent mixtures [THF/heptane (1:5) and IPAc/heptane (1:2)] afforded slurries with sticky solids on the bottom and sides of the vial. All other solvent mixtures afforded oils.

As shown in Table 3A, several solvents systems afforded solids with reduced residual heptane content compared to the starting material. However, in most cases a decrease in heptane content was accompanied by an increase in the residual solvent content of the co-solvent that was used. Some slurry solvent systems which afforded material with residual solvent content below or near the acceptable limit for each of the respective solvents were IPAc/heptane (1:2) and pure water. The IPAc/heptane slurry initially afforded sticky solids but became free flowing after adding another portion of heptane.

TABLE 3A
Slurry Study Results

| | | | | | | Appearance | | | | |
|---|---|---|---|---|---|---|---|---|---|---|
| | | | | | | after | | | | |
| | | | | Appearance | Additional | anti- | | | | wt % |
| Material | | | Solvent | after | anti- | solvent | | | | Residual |
| Amt | Solvent | Solvent | Amt | solvent | solvent | Amt | Slurry | Drying | Resid | Solvent |
| (mg) | System | ratio | (mL) | addition | addition | (mL) | Time | Temp | Solvent | (ppm) |
| — | — | — | — | — | — | — | — | — | heptane | 5.9% |
| | | | | | | | | | | (59,000) |
| 47.2 | THF/hept | 1.0:2.0 | 0.5 | oil | 0.25 | oil | — | — | — | — |
| 50.5 | THF/hept | 1.0:5.0 | 0.5 | sticky | 0.25 | sticky | — | — | — | — |
| | | | | solids | | solids | | | | |
| 44.4 | THF/hept | 1.0:10 | 1.0 | slurry | n/a | slurry | 1 day | RT | heptane | 3.4% |
| | | | | | | | | | | (34,000) |
| | | | | | | | 1 day | RT + 55 | heptane | 2.4% |
| | | | | | | | | ° C. | | (24,000) |
| | | | | | | | 7 days | RT | heptane | 2.8% |
| | | | | | | | | | | (28,000) |
| | | | | | | | 7 days | RT + 55 | heptane | 2.1% |
| | | | | | | | | ° C. | | (21,000) |
| 45.5 | Acetone/ | 1.0:2.0 | 0.5 | oil | 0.25 | oil | — | — | — | — |
| | water | | | | | | | | | |
| 49.0 | MTBE/ | 2.0:1.0 | 1.0 | slurry | n/a | slurry | 1 day | RT | heptane | 1.3% |
| | hept | | | | | | | | MTBE | (13,000) |
| | | | | | | | | | | 6.7% |
| | | | | | | | | | | (67,000) |
| | | | | | | | 1 day | RT + 55 | heptane | 0.3% |
| | | | | | | | | ° C. | MTBE | (3,400) |
| | | | | | | | | | | 2.6% |
| | | | | | | | | | | (26,000) |
| | | | | | | | 7 days | RT | heptane | 1.6% |
| | | | | | | | | | MTBE | (16,000) |
| | | | | | | | | | | 8.2% |
| | | | | | | | | | | (82,000) |
| | | | | | | | 7 days | RT + 55 | heptane | 0.3% |
| | | | | | | | | ° C. | MTBE | (2,500) |
| | | | | | | | | | | 1.8% |
| | | | | | | | | | | (18,000) |
| 46.8 | EtOH/ | 1.0:2.0 | 0.5 | oil | 0.25 | oil | — | — | — | — |
| | water | | | | | | | | | |
| 47.3 | EtOAc/ | 1.0:3.0 | 0.5 | oil | 0.5 | oil | — | — | — | — |
| | hept | | | | | | | | | |
| 52.2 | IPAc/ | 1.0:2.0 | 0.5 | sticky | 0.25 | slurry | 1 day | RT | heptane | 1.8% |
| | hept | | | solids | | | | | IPAc | (18,000) |
| | | | | | | | | | | 12.0% |
| | | | | | | | | | | (120,000) |
| | | | | | | | 1 day | RT + 55 | heptane | 0.8% |
| | | | | | | | | ° C. | IPAc | (7,900) |
| | | | | | | | | | | (26,000) |
| | | | | | | | 7 days | RT + 55 | heptane | 1.4% |
| | | | | | | | | | IPAc | (14,000) |
| | | | | | | | | | | 12.7% |
| | | | | | | | | | | (127,000) |
| | | | | | | | 7 days | RT + 55 | heptane | 0.3% |
| | | | | | | | | ° C. | IPAc | (2,900) |
| | | | | | | | | | | 0.8% |
| | | | | | | | | | | (7,700) |
| 54.3 | IPA/ | 1.0:2.5 | 0.5 | oil | 0.25 | oil | — | — | — | — |
| | water | | | | | | | | | |
| 53.4 | MEK/ | 1.0:3.0 | 0.5 | oil | 0.5 | oil | — | — | — | — |
| | hept | | | | | | | | | |
| — | — | — | — | — | — | — | — | — | heptane | 5.9% |
| | | | | | | | | | | (59,000) |
| 46.9 | toluene | 1.0:1.0 | 0.5 | slurry | n/a | slurry | 1 day | RT | heptane | 0.3% |
| | hept | | | | | | toluene | | | (3,500) |
| | | | | | | | | | | 12.1% |
| | | | | | | | | | | (121,000) |
| | | | | | | | 1 day | RT + 55 | heptane | 0.3% |
| | | | | | | | | ° C. | toluene | (3,100) |
| | | | | | | | | | | 7.7% |
| | | | | | | | | | | (77,000) |
| | | | | | | | 7 days | RT | heptane | 0.3% |
| | | | | | | | | | toluene | (3,100) |
| | | | | | | | | | | 13.0% |
| | | | | | | | | | | (130,000) |
| | | | | | | | 7 days | RT + 55 | heptane | 0.2% |
| | | | | | | | | ° C. | toluene | (2,200) |
| | | | | | | | | | | 5.6% |
| | | | | | | | | | | (56,000) |
| 55.1 | MeOH/ | 1.0:1.0 | 0.5 | oil | 0.25 | oil | — | — | — | — |
| | water | | | | | | | | | |
| 52.2 | MeCN/ | 1.0:1.5 | 0.5 | oil | 0.25 | oil | — | — | — | — |
| | water | | | | | | | | | |
| 44.3 | IPAc/ | 1.0:4.0 | 1.0 | slurry | n/a | n/a | 1 day | RT | heptane | 1.5% |
| | hept | | | | | | | | IPAc | (15,000) |
| | | | | | | | | | | 10.0% |
| | | | | | | | | | | (100,000) |
| | | | | | | | 1 day | RT + 55 | heptane | 1.2% |
| | | | | | | | | ° C. | IPAc | (12,000) |
| | | | | | | | | | | 2.5% |
| | | | | | | | | | | (25,000) |
| | | | | | | | 7 days | RT | heptane | 0.9% |
| | | | | | | | | | IPAc | (8,500) |
| | | | | | | | | | | 4.5% |
| | | | | | | | | | | (45,000) |
| 52.6 | acetone/ | 1.0:4.0 | 0.5 | oil | 0.25 | oil | — | — | — | — |
| | hept | | | | | | | | | |
| 43.9 | EtOH/ | 1.0:4.0 | 0.5 | oil | 0.25 | oil | — | — | — | — |
| | hept | | | | | | | | | |
| 50.1 | water | n/a | 1.5 | slurry | n/a | n/a | 1 day | RT | heptane | 1.3% |
| | | | | | | | | | | (13,000) |
| | | | | | | | 1 day | RT + 55 | heptane | 0.8% |
| | | | | | | | | ° C. | | (8,200) |
| | | | | | | | 4 days | RT | heptane | 1.6% |
| | | | | | | | | | | (16,000) |
| | | | | | | | 7 days | RT + 55 | heptane | 1.6% |
| | | | | | | | | ° C. | | (16,000) |
| | | | | | | | 7 days | 55 ° C. | heptane | 1.4% |
| | | | | | | | | | | (14,000) |

In addition to the slurry investigations, precipitation experiments were evaluated to decrease the residual amount of heptane in PX-866. In the first experiment, PX-866 was dissolved in toluene (0.25 mL) at ambient temperature. Heptane (0.25 mL) was then slowly added in an attempt to induce precipitation. Sticky/oily solids were formed upon the heptane addition. A small amount of seeds (about 1 mg of starting material) was then added, and the mixture was stirred with a spatula. After about 1 minute of stirring, free flowing solids were generated. After 20 hours of stirring, a sample of the slurry was taken, and the solids were isolated by centrifuge filtration and then dried at ambient temperature under vacuum overnight. A portion of the solids was then dried further at 55° C. under vacuum overnight. Both dried samples were then analyzed by 1H-NMR to determine the residual solvent content. The remainder of the slurry was allowed to continue stirring for seven days at ambient temperature. A portion was then sampled and analyzed as previously described. As shown in Table 4A, the residual heptane content significantly decreased for the samples from this slurry; however, the toluene content was very high (>50,000 ppm).

The second experiment was performed by first adding MTBE (1 mL) to PX-866 [51.5 mg] in an attempt to dissolve the starting material. Dissolution was not observed as sticky solids were formed. Acetone (0.05 mL) was then added to dissolve all solids. The resulting solution was allowed to stir at ambient temperature overnight. No solids precipitated. The solvents were then removed by evaporation under a stream of nitrogen. MTBE (0.5 mL) was added again, and sticky solids were formed. Heptane was then added (0.45 mL) which afforded the formation of free flowing solids. The slurry was allowed to continue stirring at ambient temperature for seven days at which point the solids were isolated and dried as described previously. The dried solids were then analyzed by ¹H-NMR to determine the residual solvent content. As shown in Table 4A, the residual heptane content was decreased significantly, however the MTBE content remained very high (>16,000 ppm).

TABLE 4A
Precipitation Experiments

| | | Primary | Antisol | Appearance | | Appearance | | | | Wt % |
|---|---|---|---|---|---|---|---|---|---|---|
| API | | Solvent | vent | after | | after | | Drying | | residual |
| Amt | Solvent | amount | amount | solvent | Extra | extra | Slurry | temper- | Residual | solvent |
| (mg) | systme | (mL) | (mL) | addition | addition | addition | time- | ature | Solvent | (ppm) |
| — | — | — | — | — | — | — | — | — | heptane | 5.9% |
| | | | | | | | | | | (59,000) |
| 48.6 | Toluene/ | 0.25 | 0.5 | sticky | 1 mg | slurry | 1 day | RT | heptane | 0.3% |
| | hept | | | solids | seeds | | | | toluene | (2,900) |
| | | | | | | | | | | 11.9% |
| | | | | | | | | | | (119,000) |
| | | | | | | | 1 day | RT + 55 | heptane | 0.3% |
| | | | | | | | | ° C. | toluene | (2,500) |
| | | | | | | | | | | 7.4% |
| | | | | | | | | | | (74,000) |
| | | | | | | | 7 days | RT | heptane | 0.3% |
| | | | | | | | | | toluene | (2,900) |
| | | | | | | | | | | 12.9% |
| | | | | | | | | | | (129,000) |
| | | | | | | | 7 days | RT + 55 | heptane | 0.2% |
| | | | | | | | | | toluene | (1,900) |
| | | | | | | | | ° C. | | 5.2% |
| | | | | | | | | | | (52,000) |
| 51.5 | MTBE/ | 1 | n/a | sticky | Acetone | Solution | — | — | — | — |
| | acetone | | | solids | (50 μl) | | | | | |
| 51.5 | MTBE/ | 0.5 | 0.45 | slurry | n/a | n/a | 7 days | RT | heptane | 2.8% |
| | hept | | | | | | | | MTBE | (28,000) |
| | | | | | | | | | | 7.3% |
| | | | | | | | | | | (73,000) |
| | | | | | | | 7 days | RT + 55 | heptane | 0.4% |
| | | | | | | | | ° C. | MTBE | (4,100) |
| | | | | | | | | | | 1.6% |
| | | | | | | | | | | (16,000) |

Initial attempts to find successful solvent systems for crystallization of PX-866 described above gave sub-optimal results due to high residual solvent amounts and/or lack of crystallinity and/or formation of sticky solids or oils. Extensive experimentation was needed to identify solvent systems that yielded crystalline forms of PX-866 and these additional attempts are shown in Examples 1-3.

By way of example, a stability study established improved stability of the crystalline solvates against heat and humidity compared to the stability of the amorphous material. The study was conducted under 40° C. and 75% relative humidity to be pulled at 1, 2, 3, 4 and 8 week time points for HPLC and XRPD analysis. (See Example 17 and FIG. 13). Further, it is observed that exemplary anisole and toluene solvates have a better stability over amorphous PX-866 after only one week. The propyl acetate solvate is also more stable than the amorphous material. XRPD analysis also showed that crystalline forms did not convert or undergo a physical change over the duration of the study.

FIG. 16(A), FIG. 16(B), FIG. 17(A) and FIG. 17(B) show a comparison between amorphous PX-866 and crystalline anisole solvate form of PX-866. As shown in the figures, the amorphous form of PX-866 shows small and heterogenous particle sizes and clumping of particles. The crystalline anisole solvate form shows well defined prismatic crystals and uniform particle size with no clumping of particles. The experiments described herein led to identification of certain solvate forms including a crystalline anisole solvate form which removed some disadvantages associated with the amorphous PX-866.

### Formulation of Non-Amorphous PX-866

In initial formulation studies, it was observed that amorphous forms of Compound 1 exhibited undesirable properties for formulation including rapid degradation from heat and humidity, low flowability as well as unwanted hygroscopicity which required low-moisture conditions to produce sample formulations. In some instances, the synthetic routes for the amorphous forms led to variable purity and it was observed in some cases that heptane was trapped in the last step to widely varying degrees. Dissolution studies also have shown capsules filled with amorphous Compound 1 exhibit plugging (i.e., a sticky mass) in the capsule.

Accordingly, provided herein are crystalline forms of Compound 1 and/or analogs thereof which allow for ease of formulation. In some embodiments, the crystalline forms and solvates thereof of Compound 1 and/or analogs thereof described herein have improved properties with respect to the amorphous form. In some embodiments, the crystalline forms of Compound 1 and/or analogs thereof exhibit higher stability than amorphous form. In some embodiments, the crystalline forms and solvates of compound 1 described herein do not exhibit a tendency to form a sticky mass and are easier to handle during manufacturing processes.

Further studies with crystalline forms of Compound 1 and/or analogs thereof show that these forms have better flowability than the amorphous form, do not exhibit plugging when filled in capsules, have good aqueous solubility and have a higher melting point, and better stability as described above. In some embodiments, crystalline forms of Compound 1 and/or analogs thereof show less variability in vitro dissolution profiles as compared to the amorphous form. In some instances, crystalline forms of Compound 1 and/or analogs thereof have better flowability as compared to the amorphous form.

Additionally, the crystalline forms of Compound 1 provided herein are easier to blend because of their crystal morphology. The crystalline forms also exhibit improved flowability during manufacturing processes. For example, a dry blend of PX-866 anisole solvate, mannitol and magnesium stearate has a flowability of 4 mm diameter, with a bulk density of 0.5262 g/mL and a tap density of 0.6446 g/mL. The formulations comprising crystalline forms of compound 1 show less variation in release of the active agent in vivo compared to capsules comprising amorphous PX-866. For example, Example 19e describes a 2 mg tablet formulation comprising crystalline anisole solvate of PX-866. The tablets of Example 19e pass dissolution testing specifications of no less than 80% released at 30 minutes. Clinical PK studies show a smaller coefficient of variation for PK parameters in volunteers dosed with crystalline PX-866 tablets compared to the variation in volunteers dosed with amorphous PX-866 capsules.

Further, crystalline forms of Compound 1 (PX-866) have increased purity or are substantially more pure than the amorphous form. For example, the anisole solvate form described herein has no detectable wortmannin, e.g., when tested by HPLC and/or GC.

### CERTAIN DEFINITIONS

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein, the term “about” means plus or minus 10% of the numerical value of the number with which it is being used. Therefore, about 50% means in the range of 45%-55%. “Optional” or “optionally” may be taken to mean that the subsequently described structure, event or circumstance may or may not occur, and that the description includes instances where the events occurs and instances where it does not.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a wortmannin analog or metabolite thereof, can include, but is not limited to, providing a wortmannin analog or metabolite thereof into or onto the target tissue; providing a wortmannin analog or metabolite thereof systemically to a patient by, e.g., intravenous injection whereby the therapeutic reaches the target tissue or cells. “Administering” a composition may be accomplished by injection, topical administration, and oral administration or by other methods alone or in combination with other known techniques.

As used herein, the term “therapeutic” means an agent utilized to treat, combat, ameliorate, prevent or improve an unwanted condition or disease of a patient.

In some embodiments, a therapeutic agent is directed to the treatment and/or the amelioration of or reversal of the symptoms of a cancer described herein. In some embodiments, a therapeutic agent is directed to the treatment and/or the amelioration of or reversal of the symptoms of a fibrotic condition described herein. In some embodiments, a therapeutic agent described herein is directed to treatment of pulmonary fibrosis and/or the amelioration of or reversal of the symptoms of pulmonary fibrosis.

The term “animal” as used herein includes, but is not limited to, humans and non-human vertebrates such as wild, domestic and farm animals. The terms “patient” and “subject” and “individual” are interchangeable and may be taken to mean any living organism which may be treated with compounds of the present disclosure. As such, the terms “patient” and “subject” may include, but are not limited to, any non-human mammal, any primate or a human.

The term “inhibiting” includes the administration of a compound of the present disclosure to prevent the onset of symptoms, alleviate symptoms, or eliminate the disease, condition or disorder.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology). As such, a non-limiting example of a “therapeutically effective amount” or “effective amount” of a composition of the present disclosure may be used to inhibit, block, or reverse the activation, migration, or proliferation of cells or to effectively treat cancer or ameliorate the symptoms of cancer.

“Wortmannin” is a naturally occurring compound isolated from culture broths of the fungus Penicillium wortmannin. Wortmannin irreversibly inhibits PI-3-kinase through covalent interaction with a specific lysine on the kinase: Lys⁸⁰² of the ATP binding pocket of the catalytic site of the p110α isoform or Lys⁸⁸³ of the p110δ isoform. Most isoforms of PI-3 kinase, such as p110α, p110β, p110δ and p110γ for example, are inhibited equally by wortmannin. Wortmannin demonstrates liver and hematologic toxicity, however, and is a biologically unstable molecule. Samples stored as aqueous solutions at either 37° C. or 0° C. at neutral pH are subject to decomposition by hydrolytic opening of the furan ring. It has been shown that the electrophilicity of the furan ring is central to the inhibitory activity of wortmannin. The irreversible inhibition of PI-3-kinase occurs by formation of an enamine following the attack of the active lysine of the kinase on the furan ring at position C(20) of wortmannin. Thus, decomposition of wortmannin may interfere with its inhibitory activity on PI-3 kinases.

The terms “treat,” “treated,” “treatment” or “treating” as used herein, in one embodiment, refers to therapeutic treatment. For the purposes described herein, treatment-induced beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treat,” “treated,” “treatment” or “treating” as used herein includes prophylactic or preventative measures, wherein the object is to prevent or slow (lessen) an undesired physiological condition, disorder or disease.

### Embodiments

In one aspect, provided herein is a crystalline form of a compound having a structural formula Compound 1,

[chemical structure]

and/or analog thereof which is substantially free of wortmannin. In some embodiments, the crystalline form has up to about 2% wortmannin. In certain embodiments, the crystalline form has up to about 1% wortmannin, up to about 0.5% wortmannin, up to about 0.3% wortmannin, up to about 0.1% wortmannin or up to about 0.01% wortmannin. In some embodiments, the crystalline form is free of wortmannin. In certain embodiments, the crystalline form is a toluene, anisole, cumene, propyl acetate, 4-methyl-2-pentanone, chlorobenzene, or 1-pentanol solvate of Compound 1 and/or analog thereof.

In another aspect provided herein a substantially pure crystalline form of a compound having a structural formula, Compound 1,

[chemical structure]

and/or analog thereof wherein the form is a solvate. In certain embodiments, the substantially pure crystalline solvate form is a toluene, anisole, cumene, propyl acetate, 4-methyl-2-pentanone or 1-pentanol solvate of Compound I. Such solvate forms include anisole solvate, cumene solvate, propyl acetate solvate, 4-methyl-2-pentanone solvate, chlorobenzene solvate, 1-pentanol solvate, or the like.

In some embodiments, the crystalline form of Compound 1 and/or analog thereof is an anisole solvate. In certain embodiments, the crystalline form of Compound 1 and/or analog thereof is a crystalline form prepared from a solution comprising anisole. For instance, the anisole solvate of Compound 1 and/or analog thereof is prepared from an anisole-containing supernatant, solution, dispersion or emulsion. In other embodiments, the anisole solvate is prepared from addition of anisole as antisolvent to a solution comprising Compound 1 and/or analog thereof. In some embodiments, the process of preparing the anisole solvate utilizes seeding (e.g., addition of crystals of the anisole solvate or glass powder) or via any other known processes. In other embodiments, the process of preparing the anisole solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the anisole crystalline form exhibits a predominant endotherm at about 146° C. as measured by Differential Scanning calorimeter. In some embodiments, the scan rate is 10° C. per minute. In certain embodiments, the anisole crystalline form has an X-ray powder diffraction pattern having at least two degrees 2-theta values selected from 7.9, 8.5, 10.2, 11.1, 14.0, 14.2, 17.9, 18.7, 21.0, 21.2, and 28.2±0.1. In certain embodiments, the crystalline form exhibits a single crystal X-ray crystallographic analysis at 120 K with crystal parameters as the following:

| Space Group | P2₁2₁2₁ |
|---|---|
| a, Å | 13.7140(3)° |
| b, Å | 15.4272(4) |
| c, Å | 15.6890(4) |
| α | 90 |
| β | 90 |
| γ | 90 |
| Z (molecules/unit cell) | 4° |
| Calculated Density (g/cm) | 1.268.° |

In some embodiments, the crystalline form of Compound 1 and/or analog thereof is a propyl acetate solvate. In certain embodiments, the crystalline form of Compound 1 and/or analog thereof is a crystalline form prepared from a solution comprising propyl acetate. In certain embodiments, the crystalline form exhibits a predominant endotherm at about 80.5° C. as measured by Differential Scanning calorimeter. In some of such embodiments, the scan rate is about 10° C. per minute. In certain embodiments, the crystalline form has an X-ray powder diffraction pattern having at least two degrees 2-theta values selected from 8.0, 8.4, 10.2, 11.0, 14.0 and 19.2, ±0.1. In certain embodiments, the crystalline form exhibits a single crystal X-ray crystallographic analysis at 100 K with crystal parameters as the following:

| Space Group | P2₁2₁2₁ |
|---|---|
| a, Å | 13.4963(5)° |
| b, Å | 15.5158(5) |
| c, Å | 15.6912(6) |
| α | 90 |
| β | 90 |
| γ | 90 |
| Z (molecules/unit cell) | 4° |
| Calculated Density (g/cm) | 1.269.° |

In some embodiments, the crystalline form of Compound 1 and/or analog thereof is a toluene solvate. In certain embodiments, the crystalline form of Compound 1 and/or analog thereof is a crystalline form prepared from a solution comprising toluene. In certain embodiments, the crystalline form exhibits a predominant endotherm at about 142.0° C. as measured by Differential Scanning calorimeter. In some of such embodiments, the scan rate is about 10° C. per minute. In certain embodiments, the crystalline form has an X-ray powder diffraction pattern having at least two degrees 2-theta values selected from 12.5, 14.0, and 21.1, ±0.1.

In some embodiments, the crystalline form of Compound 1 and/or analog thereof is a cumene solvate. In certain embodiments, the crystalline form of Compound 1 and/or analog thereof is a crystalline form prepared from a solution comprising cumene. In certain embodiments, the crystalline form has an X-ray powder diffraction pattern expressed in degrees 2-theta at 7.8, 8.4, 10.7, 10.1, 13.7, 14.1, 18.1, 18.9, 20.6, and 20.8, ±0.1.

In some embodiments, the crystalline form of Compound 1 and/or analog thereof is a 4-methyl-2-pentanone solvate. In certain embodiments, the crystalline form of Compound 1 and/or analog thereof is a crystalline form prepared from a solution comprising 4-methyl-2-pentanone. In certain embodiments, the crystalline form has an X-ray powder diffraction pattern expressed in degrees 2-theta at 7.9, 8.4, 10.2, 10.9, 13.9, 14.2, 18.5, 19.2, and 20.7, ±0.1.

In some embodiments, the crystalline form of Compound 1 and/or analog thereof is a 1-pentanol solvate. In certain embodiments, the crystalline form of Compound 1 and/or analog thereof is a crystalline form prepared from a solution comprising 1-pentanol. In certain embodiments, the crystalline form has an X-ray powder diffraction pattern expressed in degrees 2-theta at 8.1, 8.5, 10.2, 11.1, 12.5, 14.0, 14.3, 17.9, 18.8, 20.7, and 21.3, ±0.1.

In some embodiments, the crystalline form of Compound 1 and/or analog thereof is a chlorobenzene solvate. In certain embodiments, the crystalline form of Compound 1 and/or analog thereof is a crystalline form prepared from a solution comprising chlorobenzene. In certain embodiments, the crystalline form has an X-ray powder diffraction pattern expressed in degrees 2-theta at 8.0, 8.5, 10.3, 11.1, 14.1, 17.9, 18.8, 19.1, 21.0 and 28.3, ±0.1.

In another aspect, contemplated within the scope of embodiments provided herein are crystalline forms of PX-866 wherein the form is a co-crystal. Contemplated within the scope of such embodiments are crystalline forms of PX-866 that are co-crystals with, for example, ascorbic acid, tartaric acid, citric acid, alcohol amines, alcohol pyridines and the like.

In yet another aspect, contemplated within the scope of embodiments provided herein are crystalline forms of PX-866 wherein the form is a solvate/co-crystal. Contemplated within the scope of such embodiments are crystalline forms of PX-866 such that PX-866 solvates (e.g., anisole solvate, toluene solvate) are co-crystallized with, for example, ascorbic acid, tartaric acid, citric acid, alcohol amines, alcohol pyridines and the like.

In further aspects, contemplated within the scope of embodiments provided herein are crystalline forms of PX-866 wherein the form is a mixed solvate-hydrate, e.g., (where water and the solvent constitute components in the crystal lattice). Contemplated within the scope of such embodiments are crystalline solvate forms of PX-866 (e.g., anisole solvate) such that PX-866 solvates are solvate hydrates (e.g., anisole solvate hydrate).

In other embodiments, there are provided methods of making crystalline solvate form of a compound having a structural formula

[chemical structure]

comprising adding antisolvent to a solution, suspension, dispersion or emulsion of the compound in a solvent, where Compound 1 has differential solubility in the solvent compared to the antisolvent.

In some of such embodiments, the solvent is selected from water, acetic acid, acetone, anisole, 1-butanol, 2-butanol, butyl acetate, tert-butylmethyl ether, cumene, dimethylsulfoxide, ethanol, ethyl acetate, diethyl ether, ethyl formate, formic acid, heptanes, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, and propyl acetate, or combination thereof.

In some of such embodiments, the solvent is selected from acetonitrile, chlorobenzene, chloroform, cyclohexane, 1,2-dichloroethane, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidone, nitromethane, pyridine, sulfolane, tetrahydrofuran, tetralin, toluene, and xylene, or combination thereof.

In some of such embodiments, the antisolvent is selected from water, acetic acid, acetone, anisole, 1-butanol, 2-butanol, butyl acetate, tert-butylmethyl ether, cumene, dimethylsulfoxide, ethanol, ethyl acetate, diethyl ether, ethyl formate, formic acid, heptanes, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, and propyl acetate, or combination thereof.

In some of such embodiments, the antisolvent is selected from acetonitrile, chlorobenzene, chloroform, cyclohexane, 1,2-dichloroethane, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidone, nitromethane, pyridine, sulfolane, tetrahydrofuran, tetralin, toluene, and xylene, or combination thereof.

In other embodiments, there are provided methods of making crystalline solvate form of a compound having a structural formula

[chemical structure]

comprising adding antisolvent to a solution of the compound in THF. In some embodiments, the antisolvent is toluene, anisole, cumene, propyl acetate, 4-methyl-2-pentanone, chlorobenzene, or 1-pentanol.

In some embodiments, the crystalline form of Compound 1 and/or analog thereof is an anisole solvate. In certain embodiments, the crystalline form of Compound 1 and/or analog thereof is a crystalline form prepared from a solution comprising anisole. For instance, the anisole solvate of Compound 1 and/or analog thereof is prepared from an anisole supernatant. In other embodiments, the anisole solvate is prepared from addition of anisole as antisolvent to a solution comprising Compound 1 and/or analog thereof. In some embodiments, the process of preparing the anisole solvate utilizes seeding (e.g., addition of crystals of the anisole solvate or glass powder) or via any other known processes. In other embodiments, the process of preparing the anisole solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline form of the anisole solvate of Compound I and/or analog thereof exhibits a predominant endotherm at about 146° C. as measured by Differential Scanning calorimeter. In some of such embodiments, the scan rate is about 10° C. per minute. In certain embodiments, the crystalline form has an X-ray powder diffraction pattern having at least two degrees 2-theta values selected from 7.9, 8.5, 10.2, 11.1, 14.0, 14.2, 17.9, 18.7, 21.0, 21.2, and 28.2±0.1. In certain embodiments, the crystalline form exhibits a single crystal X-ray crystallographic analysis at 120 K with crystal parameters as the following:

| Space Group | P2₁2₁2₁ |
|---|---|
| a, Å | 13.7140(3)° |
| b, Å | 15.4272(4) |
| c, Å | 15.6890(4) |
| α | 90 |
| β | 90 |
| γ | 90 |
| Z (molecules/unit cell) | 4° |
| Calculated Density (g/cm) | 1.268.° |

In some instances, similar solvents such as toluene, cumene, chlorobenzene, o-xylene, m-xylene, p-xylene and the like are used to prepare a similar crystalline solvate.

In some embodiments, the crystalline form of Compound 1 and/or analog thereof is a propyl acetate solvate. In certain embodiments, the crystalline form of Compound 1 and/or analog thereof is a crystalline form prepared from a solution comprising propyl acetate. For instance, the propyl acetate solvate of Compound 1 and/or analog thereof is prepared from a propyl acetate supernatant. In other embodiments, the propyl acetate solvate is prepared from addition of propyl acetate as antisolvent to a solution comprising Compound 1 and/or analog thereof. In some embodiments, the process of preparing the propyl acetate solvate utilizes seeding (e.g., addition of crystals of the propyl acetate solvate or glass powder) or via any other known processes. In other embodiments, the process of preparing the propyl acetate solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline propyl acetate solvate of Compound 1 and/or analog thereof exhibits a predominant endotherm at about 80.5° C. as measured by Differential Scanning calorimeter. In some of such embodiments, the scan rate is about 10° C. per minute. In certain embodiments, the crystalline propyl acetate solvate has an X-ray powder diffraction pattern having at least two degrees 2-theta values selected from 8.0, 8.4, 10.2, 11.0, 14.0 and 19.2, ±0.1. In certain embodiments, the crystalline propyl acetate solvate exhibits a single crystal X-ray crystallographic analysis at 100 K with crystal parameters as the following:

| Space Group | P2₁2₁2₁ |
|---|---|
| a, Å | 13.4963(5)° |
| b, Å | 15.5158(5) |
| c, Å | 15.6912(6) |
| α | 90 |
| β | 90 |
| γ | 90 |
| Z (molecules/unit cell) | 4° |
| Calculated Density (g/cm) | 1.269.° |

In some embodiments, other similar solvents such as methyl acetate, ethyl acetate, isopropyl acetate, butyl acetate, and the like are used to prepare a similar crystalline solvate.

In some embodiments, the crystalline form of Compound 1 and/or analog thereof is a toluene solvate. In certain embodiments, the crystalline form of Compound 1 and/or analog thereof is a crystalline form prepared from a solution comprising toluene. For instance, the toluene solvate of Compound 1 and/or analog thereof is prepared from a toluene supernatant. In other embodiments, the toluene solvate is prepared from addition of toluene as antisolvent to a solution comprising Compound 1 and/or analog thereof. In some embodiments, the process of preparing the toluene solvate utilizes seeding (e.g., addition of crystals of the toluene solvate or glass powder) or via any other known processes. In other embodiments, the process of preparing the toluene solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline toluene solvate exhibits a predominant endotherm at about 142.0° C. as measured by Differential Scanning calorimeter. In some of such embodiments, the scan rate is about 10° C. per minute. In certain embodiments, the crystalline toluene solvate has an X-ray powder diffraction pattern having at least two 2-theta values selected from 12.5, 14.0, and 21.1, ±0.1.

In some embodiments, the crystalline form of Compound 1 and/or analog thereof is a cumene solvate. In certain embodiments, the crystalline form is a crystalline form of Compound 1 and/or analog thereof prepared from a solution comprising cumene. For instance, the cumene solvate of Compound 1 and/or analog thereof is prepared from a cumene supernatant. In other embodiments, the cumene solvate is prepared from addition of cumene as antisolvent to a solution comprising Compound 1 and/or analog thereof. In some embodiments, the process of preparing the cumene solvate utilizes seeding (e.g., addition of crystals of the cumene solvate or glass powder) or via any other known processes. In other embodiments, the process of preparing the cumene solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline cumene solvate has an X-ray powder diffraction pattern expressed in degrees 2-theta at 7.8, 8.4, 10.1, 10.7, 13.7, 14.1, 18.1, 18.9, 20.6, and 20.8, ±0.1.

In some embodiments, the crystalline form of Compound 1 and/or analog thereof is a chlorobenzene solvate. In certain embodiments, the crystalline form of Compound 1 and/or analog thereof is a crystalline form prepared from a solution comprising chlorobenzene. In certain embodiments, the crystalline chlorobenzene solvate has an X-ray powder diffraction pattern expressed in degrees 2-theta at 8.0, 8.5, 10.3, 11.1, 14.1, 17.9, 18.8, 19.1, 21.0 and 28.3, ±0.1.

In some embodiments, the crystalline form of Compound 1 and/or analog thereof is a 4-methyl-2-pentanone solvate. In certain embodiments, the crystalline form is a crystalline form of Compound 1 and/or analog thereof prepared from a solution comprising 4-methyl-2-pentanone. For instance, the 4-methyl-2-pentanone solvate of Compound I and/or analog thereof is prepared from a 4-methyl-2-pentanone supernatant. In other embodiments, the 4-methyl-2-pentanone solvate is prepared from addition of 4-methyl-2-pentanone as antisolvent to a solution comprising Compound 1 and/or analog thereof. In some embodiments, the process of preparing the 4-methyl-2-pentanone solvate utilizes seeding (e.g., addition of crystals of the 4-methyl-2-pentanone solvate or glass powder) or via any other known processes. In other embodiments, the process of preparing the 4-methyl-2-pentanone solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline 4-methyl-2-pentanone solvate has an X-ray powder diffraction pattern expressed in degrees 2-theta at 7.9, 8.4, 10.2, 10.9, 13.9, 14.2, 18.5, 19.2, and 20.7, ±0.1.

Similarly, in some instances, other ketone solvents such as acetone, 2-butanone, and the like are used to prepare a similar crystalline solvate.

In other embodiments, the crystalline form of Compound 1 and/or analog thereof is a 1-pentanol solvate. In certain embodiments, the crystalline form of Compound 1 and/or analog thereof is a crystalline form prepared from a solution comprising 1-pentanol. For instance, the 1-pentanol solvate of Compound 1 and/or analog thereof is prepared from a 1-pentanol supernatant. In other embodiments, the 1-pentanol solvate is prepared from addition of 1-pentanol as antisolvent to a solution comprising Compound 1 and/or analog thereof. In some embodiments, the process of preparing the 1-pentanol solvate utilizes seeding (e.g., addition of crystals of the 1-pentanol solvate or glass powder) or via any other known processes. In other embodiments, the process of preparing the 1-pentanol solvate does not use seeding. Typically, the crystalline form is dried over a flow of nitrogen or under vacuum at room temperature or raised temperature (e.g. 40° C.). The crystalline form is determined by XRPD, DSC, single crystal X-ray crystallography and/or other suitable instrumental analysis.

In certain embodiments, the crystalline 1-pentanol solvate has an X-ray powder diffraction pattern expressed in degrees 2-theta at 8.1, 8.5, 10.2, 11.1, 12.5, 14.0, 14.3, 17.9, 18.8, 20.7, and 21.3, ±0.1.

Similarly, in some embodiments, other alcohol solvents such as ethanol, isopropyl alcohol, 1-butanol, t-butanol, methanol, isopentanol, glycerol, 1-octanol, 2,2,2-trifluoroethanol, and the like are used to prepare a similar crystalline solvate.

In some embodiments provide herein methods of making a crystalline solvate form of a compound having a structural formula

[chemical structure]

and/or analog thereof comprising adding antisolvent to a solution of the compound in THF. In some embodiments, the antisolvent is a benzene like solvent such as toluene, anisole, cumene, xylene, chlorobenzene, or the like. In some embodiments, the antisolvent is an ester type solvent such as methyl acetate, ethyl acetate, propyl acetate, butyl acetate, or the like. In some embodiments, the antisolvent is a ketone type solvent such as acetone, 2-butanone, 2-pentanone, 3-pentanone, 3-methyl-2-pentanone, 4-methyl-2-pentanone, or the like. In some embodiments, the antisolvent is an alcohol type solvent such as methanol, ethanol, propanol, butanol, pentanol, or the like. In certain embodiments, the antisolvent is toluene, anisole, cumene, propyl acetate, 4-methyl-2-pentanone, chlorobenzene, or 1-pentanol.

In some embodiments, provided herein are methods of making a crystalline form of a compound having a structural formula

[chemical structure]

and/or analog thereof that is substantially free of wortmannin, comprising cooling down a supernatant, solution, suspension, dispersion or emulsion of the compound to 4° C. to −20° C. In some embodiments, the supernatant, solution, suspension, dispersion or emulsion is prepared from an ester type solvent such as methyl acetate, ethyl acetate, propyl acetate, butyl acetate, or the like. In some embodiments, the supernatant, solution, suspension, dispersion or emulsion is prepared from a ketone type solvent such as acetone, 2-butanone, 2-pentanone, 3-pentanone, 3-methyl-2-pentanone, 4-methyl-2-pentanone, or the like. In some embodiments, the supernatant, solution, suspension, dispersion or emulsion is prepared from an alcohol type solvent such as methanol, ethanol, propanol, butanol, pentanol, or the like. In certain embodiments, the supernatant, solution, suspension, dispersion or emulsion comprises toluene, anisole, cumene, propyl acetate, 4-methyl-2-pentanone, chlorobenzene, or 1-pentanol.

### Compound 1 Analogs

In some embodiments, analogs of Compound 1 include compounds of Formula IA or IB:

[chemical structure]

wherein:
- - - is an optional bond;
n is 1-6;
Y is a heteroatom
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

In some embodiments, analogs of Compound 1 include compounds of formula:

[chemical structure]

wherein Y is a heteroatom and R¹ and R² are independently selected from an unsaturated alkyl, non-linear alkyl, cyclic alkyl, and substituted alkyl.

In certain embodiments of compounds of formula HA or IIB, Y is a heteroatom selected from nitrogen and sulfur and R¹ and R² are independently selected from an unsaturated alkyl, cyclic alkyl, or R¹ and R² together with Y form a heterocycle.

In yet further embodiments, an analog of Compound 1 is Acetic acid 6-hydroxy-1α-methoxymethyl-10β,13β-dimethyl-3,7,17-trioxo-4-pyrrolidin-1-methylene-1,3,4,7,10, 11β,12,13,14α,15,16,17-dodecahydro-2-oxa-cyclopenta[a]phenanthren-11-yl (PX-867) having the structure,

[chemical structure]

In additional embodiments, of Compound 1 include compounds selected from, but not limited to, PX-868, PX-870, PX-871, PX-880, PX-881, PX-882, PX-889, PX-890, DJM2-170, DJM2-171, DJM2-177, DJM2-181 and combinations thereof. In some embodiments, wortmannin analogs described herein include compounds described in GB Pat. No. 2302021, which compounds are incorporated herein by reference.

In certain embodiments, analogs of Compound 1 are 17-hydroxy (17-OH) derivatives. In some embodiments, the analog of Compound 1 is a 17-hydroxy (17-OH) derivative of PX-866. In other embodiments, the analog of Compound 1 is a 17-hydroxy (17-OH) derivative of PX-867.

In some instances the 17-hydroxy (17-OH) derivative has the following structural formula:

[chemical structure]

In other instances the 17-hydroxy (17-OH) derivative has the following structural formula:

[chemical structure]

### Pharmaceutical Composition/Formulation

In some embodiments, the crystalline solvates of Compound 1 and/or analog thereof described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a crystalline solvate of Compound 1 and/or analog thereof described herein and a pharmaceutically acceptable diluent(s), excipient(s), or carrier(s). In certain embodiments, the crystalline solvates described herein are administered as pharmaceutical compositions in which a crystalline solvate (e.g., an anisole solvate) described herein is mixed with other active ingredients, as in combination therapy. Encompassed herein are all combinations of actives set forth in the combination therapies section below and throughout this disclosure.

A pharmaceutical composition, as used herein, refers to a mixture of a crystalline solvate of Compound 1 and/or analog thereof described herein with other chemical components, such as carriers, stabilizers, diluents, dispersing agents, suspending agents, thickening agents, and/or excipients. In certain embodiments, the pharmaceutical composition facilitates administration of the crystalline solvates to an organism. In some embodiments, practicing the methods of treatment or use provided herein, therapeutically effective amounts of a crystalline solvate described herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the crystalline solvate used and other factors. The crystalline solvates described herein are used singly or in combination with one or more therapeutic agents as components of mixtures.

In such a composition, the pharmacologically active component is known as the “active ingredient”. In making the compositions, the active ingredient (e.g., a crystalline solvate) will usually be mixed with a carrier, or diluted by a carrier, or enclosed within a carrier that may be in the form of a capsule, sachet, paper or other container. When the carrier serves as a diluent, it may be a solid, semisolid, or liquid material that acts as a vehicle, excipient of medium for the active ingredient. Thus, the composition can be in the form of tablets, pills, powders, lozenges, sachets, cachets, elixirs, emulsions, solutions, syrups, suspensions, soft and hard gelatin capsules, sterile injectable solutions, and sterile packaged powders.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, talc, magnesium stearate, water, and mineral oil. The compositions can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Contemplated within the scope of embodiments presented herein are size grades of various excipients. By way of example, Example 19e shows tablet formulations comprising Pearlitol having 100 micron average particle size. Contemplated are other particle sizes for excipients (e.g., Pearlitol of 200 micron particle size) to allow for sieving thorough a sieve of a larger size and thus leaving larger crystals in the tablets. Larger crystals would allow for decreased surface area to mass ratio with larger diameter particles, thus allowing for modification of release profiles.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a crystalline solvate as described herein is administered in a local rather than systemic manner, for example, via injection of the crystalline solvate directly into an organ, often in a depot preparation or sustained release formulation.

In certain instances, the local delivery therapeutically effective amounts of a crystalline solvate of Compound 1 and/or analog thereof for the treatment of a fibrosis (e.g., pulmonary fibrosis) or a cancer can be by a variety of techniques that administer the crystalline solvate at or near the fibrotic or cancerous site. Examples of local delivery techniques include, but are not limited to, local delivery catheters, site specific carriers, implants, direct injection, or direct applications.

Another example is the delivery of a crystalline solvate of Compound 1 and/or analog thereof by polymeric endoluminal sealing. This technique employs a catheter to apply a polymeric implant to the interior surface of the lumen. The crystalline solvate incorporated into the biodegradable polymer implant is thereby released at the surgical site. One example of this delivery is described in PCT WO 90/01969 (Schindler, Aug. 23, 1989).

A further example of local delivery by an implant is by direct injection of vesicles or microparticulates into the site. These microparticulates may be composed of substances such as proteins, lipids, carbohydrates or synthetic polymers. These microparticulates have the therapeutic agent incorporated throughout the microparticle or over the microparticle as a coating. Delivery systems incorporating microparticulates are described in Lange, Science 249:1527-1533 (1990) and Mathiowitz et al, J. App. Poly. Sci., 26:809 (1981).

Local delivery by site specific carriers describes attaching the crystalline solvate of Compound 1 and/or analog thereof to a carrier which will direct the drug to the target organ. Examples of this delivery technique include the use of carriers such as a protein ligand or a monoclonal antibody.

In some embodiments, long acting formulations are administered by implantation (for example subcutaneously or intramuscularly) or by intramuscular injection. Furthermore, in other embodiments, the crystalline solvate of Compound 1 and/or analog thereof is delivered in a targeted drug delivery system, for example, in a liposome coated with organ-specific antibody. In such embodiments, the liposomes are targeted to and taken up selectively by the organ. In yet other embodiments, the crystalline solvate as described herein is provided in the form of a rapid release formulation, in the form of an extended release formulation, or in the form of an intermediate release formulation. In yet other embodiments, the crystalline solvate described herein is administered topically.

In another embodiment, the crystalline solvates of Compound 1 and/or analog thereof described herein are formulated for oral administration. In various embodiments, the crystalline solvates described herein are formulated in oral dosage forms that include, by way of example only, tablets, powders, pills, dragees, capsules, liquids, gels, syrups, elixirs, slurries, suspensions and the like.

For oral administration, the crystalline solvates of Compound 1 and/or analog thereof can be admixed with carriers and diluents, molded into tablets, or enclosed in gelatin capsules.

In certain embodiments, pharmaceutical preparations for oral use are obtained by mixing one or more solid excipient with the crystalline solvates described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of the crystalline solvates of Compound 1 and/or analog thereof described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain the crystalline solvate that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the crystalline solvates of Compound 1 and/or analog thereof described herein are formulated for buccal or sublingual administration. Formulations suitable for buccal or sublingual administration include, by way of example only, tablets, lozenges, or gels.

In still other embodiments, the crystalline solvates of Compound 1 and/or analog thereof described herein are formulated for parental injection, including formulations suitable for bolus injection or continuous infusion. The crystalline solvates described herein can alternatively be dissolved in liquids such as 10% aqueous glucose solution, isotonic saline, sterile water, or the like, and administered intravenously or by injection.

In specific embodiments, formulations for injection are presented in unit dosage form (e.g., in ampoules) or in multi-dose containers. Preservatives are, optionally, added to the injection formulations. In still other embodiments, the pharmaceutical composition of the crystalline solvates of Compound 1 and/or analog thereof described herein are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, the crystalline forms of Compound 1 and/or analog thereof described herein are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes crystalline forms described herein to be injected. In general, automatic injectors include a reservoir for holding the solution, which is in fluid communication with a needle for delivering the drug, as well as a mechanism for automatically deploying the needle, inserting the needle into the patient and delivering the dose into the patient.

In still other embodiments, the crystalline forms of Compound 1 and/or analog thereof are administered topically. The crystalline forms described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the crystalline forms of Compound 1 and/or analog thereof described herein are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the crystalline form described herein is accomplished by means of iontophoretic patches and the like. In certain embodiments, transdermal patches provide controlled delivery of crystalline forms described herein. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of the crystalline form herein; (2) a penetration enhancer; and (3) an aqueous adjuvant. In addition, transdermal formulations can include additional components such as, but not limited to, gelling agents, creams and ointment bases, and the like. In some embodiments, the transdermal formulation further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the crystalline forms of Compound 1 and/or analog thereof described herein are formulated for administration by inhalation. Various forms suitable for administration by inhalation include, but are not limited to, aerosols, mists or powders. Pharmaceutical compositions of the crystalline forms described herein are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated by reference. Formulations, which include the crystalline forms described herein, employ benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

In still other embodiments, the crystalline forms of Compound 1 and/or analog thereof described herein are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising the crystalline forms of Compound 1 and/or analog thereof described herein may be manufactured in a conventional manner, such as, by way of example only, by means of conventional mixing, dissolving, granulating, dragee-making, levigating, emulsifying, encapsulating, entrapping or compression processes.

Methods for the preparation of compositions comprising the crystalline forms of Compound 1 and/or analog thereof described herein include formulating the crystalline forms with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The formulations of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising the crystalline forms of Compound 1 and/or analog thereof described herein illustratively takes the formulation of a liquid where the agents are present in solution, in suspension or both. Typically when the composition is administered as a solution or suspension a first portion of the agent is present in solution and a second portion of the agent is present in particulate formulation, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of the crystalline forms described of Compound 1 and/or analog thereof herein. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, a crystalline form of Compound 1 and/or analog thereof described herein is present in a unit dosage form at an amount of about 0.1 to 20 mg. In certain embodiments, the crystalline form described herein is present in a unit dosage form at an amount of about 0.5 to 15 mg, about 0.5 to 10 mg, or about 1.0 to 10 mg. In some instances the crystalline form described herein is present in a unit dosage form at an amount of about 1 mg, of about 2 mg, of about 3 mg, of about 4 mg, of about 5 mg, of about 6 mg, of about 7 mg, of about 8 mg, of about 9 mg, of about 10 mg, of about 11 mg, or of about 12 mg.

In certain instances, the crystalline form described herein is administered as one or more doses amounting to between about 1 mg to about 20 mg per day. In certain instances, the crystalline form described herein is administered as one or more doses amounting to between about 1 mg to about 10 mg per day. In certain instances, the crystalline form described herein is administered as one or more doses amounting to between about 5 mg to about 20 mg per day. In certain instances, the crystalline form described herein is administered as one or more doses amounting to about 8 mg per day. In certain instances, the crystalline form described herein is administered as one or more doses amounting to about 10 mg per day. In certain instances, the crystalline form described herein is administered as one or more doses amounting to about 12 mg per day.

In some cases, for any of the embodiments described above or below, administration of a composition comprising a crystalline form of PX-866 to a patient is carried out on a continuous dosing schedule. In other cases, for any of the embodiments described above or below, administration of a composition comprising a crystalline form of PX-866 to a patient is carried out on an intermittent dosing schedule.

### Methods of Treatment

In some embodiments, provided herein are methods of treating cancer comprising administering to a subject in need thereof a crystalline form of Compound 1 and/or analog thereof described herein or a pharmaceutical composition comprising the same. In certain embodiments, the cancer is selected from the group consisting of breast cancer, lung cancer, head and neck cancer, brain cancer, abdominal cancer, colon cancer, colorectal cancer, esophageal cancer, parapharyngeal cancer, gastrointestinal cancer, glioma, liver cancer, tongue cancer, neuroblastoma, osteosarcoma, ovarian cancer, renal cancer, pancreatic cancer, retinoblastoma, cervical cancer, uterine cancer, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer including castration resistant forms, ovarian cancer including mucinous ovarian carcinoma, squamous cell carcinoma of the head and neck, colorectal carcinoma, glioblastoma, cervical carcinoma, endometrial carcinoma, gastric carcinoma, pancreatic carcinoma, leiomyosarcoma, breast carcinoma, adenocystic carcinoma, neuroendocrine tumors, brain tumors, cancer of the central nervous system, glioblastoma, and blastomas. In certain embodiments, the cancer is head and neck cancer, lung cancer, colon cancer or prostate cancer.

Accordingly, the methods and compositions comprising the crystalline solvate provided herein reduce, reverse, or delay progression and/or onset of a cancer described herein. In some embodiments, administration of the crystalline solvate described herein to an individual in need thereof reduces tumor size, thereby reducing, reversing, or delaying progression and/or onset of a cancer described herein.

In some embodiments, the methods of treating cancer described herein further comprise administering an anti-cancer agent. For any of the embodiments described above or below, a composition comprising a crystalline anisole form of PX-866 having the XRPD shown in FIG. 1 is administered to a patient in combination with a second anticancer agent.

FIG. 15 shows that in human patients, amorphous and crystalline anisole solvate forms of PX-866 show similar plasma profiles. PX-866 has shown efficacy in human trials as described in WO 2011/153488 and WO 2011/153495 international application publications, which disclosure is incorporated herein by reference.

Examples of anti-cancer agents suitable for use in combination with the crystalline PX-866 forms described herein (e.g., anisole solvate) include and are not limited to methotrexate (RHEUMATREX®, Amethopterin) cyclophosphamide (CYTOXAN®), thalidomide (THALIDOMID®), acridine carboxamide, Actimid®, actinomycin, 17-N-allylamino-17-demethoxygeldanamycin, aminopterin, amsacrine, anthracycline, antineoplastic, antineoplaston, 5-azacytidine, azathioprine, BL22, bendamustine, biricodar, bleomycin, bortezomib, bryostatin, busulfan, calyculin, camptothecin, capecitabine, carboplatin, cetuximab, chlorambucil, cisplatin, cladribine, clofarabine, cytarabine, dacarbazine, dasatinib, daunorubicin, decitabine, dichloroacetic acid, discodermolide, docetaxel, doxorubicin, epirubicin, epothilone, eribulin, estramustine, etoposide, exatecan, exisulind, ferruginol, floxuridine, fludarabine, fluorouracil, fosfestrol, fotemustine, ganciclovir, gemcitabine, hydroxyurea, IT-101, idarubicin, ifosfamide, imiquimod, irinotecan, irofulven, ixabepilone, laniquidar, lapatinib, lenalidomide, lomustine, lurtotecan, mafosfamide, masoprocol, mechlorethamine, melphalan, mercaptopurine, mitomycin, mitotane, mitoxantrone, nelarabine, nilotinib, oblimersen, oxaliplatin, PAC-1, paclitaxel, pemetrexed, pentostatin, pipobroman, pixantrone, plicamycin, procarbazine, proteasome inhibitors (e.g., bortezomib), raltitrexed, rebeccamycin, Revlimid®, rubitecan, SN-38, salinosporamide A, satraplatin, streptozotocin, swainsonine, tariquidar, taxane, tegafur-uracil, temozolomide, testolactone, thioTEPA, tioguanine, topotecan, trabectedin, tretinoin, triplatin tetranitrate, tris(2-chloroethyl)amine, troxacitabine, uracil mustard, valrubicin, vinblastine, vincristine, vinorelbine, vorinostat, zosuquidar, or the like.

In some embodiments, a composition comprising a crystalline form of PX-866 (e.g., PX-866 anisole solvate) is administered to an individual in need thereof in combination with cetuximab. In some embodiments, a composition comprising a crystalline form of PX-866 (e.g., PX-866 anisole solvate) is administered to an individual in need thereof in combination with docetaxel.

The combination therapies described herein treat various stages of cancer including stages which are locally advanced, metastatic and/or recurrent. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage 1V). Cancer designated as recurrent generally is defined as the cancer has recurred, usually after a period of time, after being in remission or after a tumor has visibly been eliminated. Recurrence can either be local, i.e., appearing in the same location as the original, or distant, i.e., appearing in a different part of the body. In certain instances, a cancer treatable by combination therapies described herein is unresectable, or unable to be removed by surgery. In further instances, a cancer treatable by the combination therapies described herein is incurable, i.e., not treatable by current treatment methods.

In some embodiments, the combination therapies described herein are administered as a first-line or primary therapy. Other subjects suitable for treatment by the combination therapies described herein include those that have completed first-line anti-cancer therapy. First-line anti-cancer therapies include chemotherapy, radiotherapy, immunotherapy, gene therapy, hormone therapy, surgery or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Chemotherapies for first-line and subsequent therapy and/or combination therapy include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, cabazitaxel, paclitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors.

Radiotherapies for first-line and subsequent therapy and/or combination therapy include factors that cause DNA damage and include what are commonly known as γ-rays, X-rays, and/or the directed delivery of radioisotopes to tumor cells. Other forms of DNA damaging factors include microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapies generally rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, an tumor antigen may stimulate a subject's immune system to target the specific tumor cells using cytotoxic T cells and NK cells. Immunotherapies include Sipuleucel-T (Provenge®), bevacizumab and the like.

A gene therapy includes a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene).

Surgery of some type is performed for resectable cancers. Surgery types include preventative, diagnostic or staging, curative and palliative surgery and can be performed as a first-line and subsequent therapy.

In some embodiments, the combination therapies described herein are administered as a second-line therapy after a first-line therapy becomes ineffective or the cancer is recurrent. In other embodiments, the combination therapies described herein administered as a third-line therapy after the first- and second-line therapy fails. In further embodiments, individuals are preselected for having completed a first- or second-line therapy. In some instances, the combination therapies described herein are administered to patients for whom prior platinum-based therapy has failed. In other instances, the combination therapies described herein are administered to patients for whom prior irinotecan therapy has failed.

Subjects, in some embodiments, can also be prescreened or preselected for sensitivity and/or effectiveness of the combination therapies described herein. A subject can be examined for certain biomarkers that allow the subject to be amenable to a combination therapy. For example, biomarkers such as phosphatase and tensin homolog (PTEN) mutations and activating mutations of PI-3K catalytic subunits may increase sensitivity to the combination therapies described herein whereas other mutations such as Ras pathway mutations may decrease sensitivity. In some embodiments, a subject is preselected based on, for example, PTEN mutational status, PTEN copy number, PI3K gene amplification, PI3K catalytic subunit alpha (PIK3CA) mutational status, K-ras mutational

Also provided herein are methods for treatment of fibrotic conditions and/or fibrosing syndromes comprising administering to a subject in need thereof a crystalline form of Compound 1 and/or analog thereof described herein or a pharmaceutical composition comprising the same.

In some embodiments, the fibrosing condition is mild, moderate or severe pulmonary fibrosis, cystic fibrosis, ocular fibrosis (e.g., scarring post glaucoma filtration surgery), endomyocardial fibrosis, mediastinal fibrosis, myelofibrosis, osteofibrosis, fibrosing colonoapathy, retroperitoneal fibrosis, interstitial pneumonia, progressive massive fibrosis in lungs, keloids, scleroderma, hypertrophic scarring, renal fibrosis, intestinal fibrosis, liver fibrosis, fibrosing cholestatic hepatitis, nephrogenic systemic fibrosis, fibrosis associated with organ transplantation, multifocal fibrosclerosis, or anaphylactic shock fibrosis. PX-866 has shown efficacy in pulmonary fibrosis as described in WO 2010/118250, which disclosure is incorporated herein by reference.

In some embodiments, the fibrotic condition is mild, moderate or severe idiopathic pulmonary fibrosis. In some embodiments, the fibrotic condition is pulmonary fibrosis associated with asbestosis, cystic fibrosis, infection, exposure to environmental allergens, lung transplant, autoimmune disease, or the fibrotic condition is drug-induced pulmonary fibrosis. In some embodiments, the fibrosing syndrome is associated with organ transplant.

### EXAMPLES

### HPLC Method

### Instrument: Agilent HP1100

### Detector: UV 254 nm

### Column: Phenomenex Luna C18 (2), 3 μm, 4.6×150 mm

### Temperature: 25° C.

Mobile Phase A: 10 mM ammonium formate in 80% water 20% ACN, adjusted to pH 4.2 with formic acid
Mobile Phase B: acetonitrile
Flow Rate: 1.0 mL/min
Injection volume: 5 μL (with ACN needle wash)

### Detection Time: 30 min

### Run Time: 38 min

Sample Preparation: dilution in acetonitrile

### Gradient:

| time (min) | % A | % B |
|---|---|---|
| 0 | 100 | 0 |
| 6 | 79 | 21 |
| 16 | 79 | 21 |
| 25 | 6 | 94 |
| 30 | 6 | 94 |
| 31 | 100 | 0 |
| 38 | 100 | 0 |

XRPD analyses were performed using an Inel XRG-3000 diffractometer equipped with a CPS (Curved Position Sensitive) detector with a 2θ range of 120°. Real time data were collected using Cu—Kα radiation at a resolution of 0.03° 29. The tube voltage and amperage were set to 40 kV and 30 mA, respectively. The monochromator slit was set at 5 mm by 160 μm. The pattern is displayed from 2.5-40° 2θ. Each sample was prepared for analysis by packing it into a thin-walled glass capillary. The capillary was mounted onto a goniometer head that is motorized to permit spinning of the capillary during data acquisition. The samples were analyzed for 5 min. Instrument calibration was performed using a silicon reference standard.

Differential scanning calorimetry (DSC) was performed using a TA Instruments Q2000 differential scanning calorimeter. Temperature calibration was performed using NIST traceable indium metal. The sample use placed into an aluminum DSC pan, and the weight was accurately recorded. The pan was covered with a lid, and the lid was crimped. A weighed, crimped aluminum pan was placed on the reference side of the cell. The sample cell was equilibrated at −30° C. and heated under a nitrogen purge at a rate of 10° C./minute, up to a final temperature of 250° C.

### Example 1

### Solubility Screening Test I

For each solvent, a 100 μL glass crimp-top vial was charged with ˜10 mg of PX-866 and sealed. By syringe, 2 parts of the appropriate solvent was added. If PX-866 dissolved completely, no further work was done. If necessary, more solvent was added to obtain a supernatant. The crimped vial was sonicated for 15 minutes and then centrifuged for 30 seconds. 1.0 μL of the supernatant was removed by syringe and added to a sealed HPLC vial containing 500 μL of acetonitrile. The same saturated solution was then heated for 30 minutes to a set point of 50° C., centrifuged again, and a second HPLC vial was prepared with 1.0 μL of the supernatant at 50° C. In the case of toluene, anisole, o-xylene, propyl acetate, 4-methyl-2-pentanone, and 1-pentanol the room temperature supernatant was transferred to a clean vial and cooled down to 0° C., and then −20° C. For each temperature, an HPLC vial was prepared with 1.0 μL of the supernatant. HPLC samples were run using the method described below and the 254 nm AUC was plotted on a calibration curve to obtain solubility data.

Screening on the first group of solvents showed that PX-866 was very soluble in most of the common solvents. Furthermore, for the solvents with intermediate solubility, increasing the temperature from room temperature to 50° C. had little to no effect on solubility. The alkanes were all good antisolvents, but appeared to be absorbed and retained by the amorphous solid in the same way as the heptane antisolvent in the supplied process. Upon storing the solubility samples at −20° C., the compound oiled out in the case of toluene and isopropyl alcohol (IPA), which suggested that these solutions might have been supersaturated.

The solubility of wortmannin in toluene, anisole, and o-xylene was also investigated to ensure that wortmannin could be excluded in a crystallization procedure. In each case, the solubility was low; however, the conversion of wortmannin to PX-866 was always close enough to quantitative that it was not a concern.

TABLE 1
PX-866 solubility Screening I

| | b.p. | Solubility | | |
|---|---|---|---|---|
| Solvent | (° C.) | (mg/mL) | | Comments |
| ethyl acetate | 77.1 | >500 @ RT | | Highly soluble |
| isopropyl acetate | 89 | >500 @ RT | | Highly soluble |
| methyl t-butyl ether | 55.2 | RT | 41.0 | Moderately soluble |
| 2-butanone | 79.6 | >500 @ RT | | Highly soluble |
| acetone | 56.5 | >500 @ RT | | Highly soluble |
| ethanol | 78.4 | >500 @ RT | | Highly soluble, product oiled out after |
| | | | | cooling overnight at −20° C. |
| isopropyl alcohol | 82.3 | >500 @ RT | | Highly soluble, but tends to oil out at RT |
| 1-butanol | 117.8 | >500 @ RT | | Highly soluble, product oiled out after |
| | | | | cooling overnight at −20° C. |
| t-Butanol | 82.4 | 30° C. | 340.0 | Partially soluble, unfavorable due to its |
| | | | | melting point (25° C.) |
| heptanes | 98.5 | RT | 0.88 | Effectively insoluble |
| 2,2,4-trimethylpentane | 99.3 | RT | 0.37 | Effectively insoluble |
| pentane | 36.1 | RT | 0.79 | Effectively insoluble |
| dimethylsulfoxide | 189 | RT | 252.5 | Partially soluble |
| methyltetrahydrofuran | 80 | >500 @ RT | | Highly soluble |
| water | 100 | RT | 3.5 | Slightly soluble, significant decomposition |
| | | | | observed by HPLC |
| anisole | 152 | RT | 9.9 | Supersaturated solution in 2 parts |
| | | 0° C. | 7.0 | Slightly soluble, data obtained from |
| | | −20° C. | 6.2 | supernatant concentration after |
| | | | | crystallization |
| diethyl ether | 34.6 | RT | 9.5 | Slightly soluble |
| toluene | 110.6 | RT | 5.2 | Supersaturated solution in 2 parts |
| | | 0° C. | 2.7 | Slightly soluble, data obtained from |
| | | −20° C. | 2.6 | supernatant concentration after |
| | | | | crystallization |
| methanol | 64.7 | >500 @ RT | | Highly soluble |
| 1,4-dioxane | 101.1 | >500 @ RT | | Highly soluble |
| hexanes | 69 | RT | 0.31 | Effectively insoluble |
| N-methyl-2-pyrrolidone | 203 | >500 @ RT | | Highly soluble |
| dimethylformamide | 153 | >500 @ RT | | Highly soluble |
| tetrahydrofuran | 66 | >500 @ RT | | Highly soluble |
| dichloromethane | 40 | >500 @ RT | | Highly soluble |
| m-xylene | 137 | RT | 4.5 | Slightly soluble |
| o-xylene | 143 | RT | 5.5 | Supersaturated solution in 2 parts |
| | | 0° C. | 7.3 | Slightly soluble, data obtained from |
| | | −20° C. | 5.7 | supernatant concentration after |
| | | | | crystallization |
| p-xylene | 137 | RT | 1.0 | Effectively insoluble |
| sec-butyl ether | 121 | RT | 1.3 | Effectively insoluble |
| diisopropyl ether | 65 | RT | 4.2 | Effectively insoluble |
| dibutyl ether | 142 | RT | 3.1 | Effectively insoluble |

### Example 2

### Crystallization Test I

The first single solvent systems targeted were those with intermediate solubility from table 1: MTBE and DMSO. PX-866 was dissolved in a minimal amount of solvent at RT and cooled to −20° C. for several days. Glass powder was introduced for nucleation. These attempts were unsuccessful.

Some of the solvents which caused the compound to oil out at −20° C. (toluene, IPA) were also investigated as single solvent systems. PX-866 was dissolved in a minimal amount of solvent and the solutions were added to new vials containing glass powder. In the case of toluene, the compound crystallized on the syringe tip and on the walls of the vial. This solid appeared crystalline by microscope. With IPA, the compound crystallized upon cooling but oiled out upon warming back to RT.

Given the positive result with toluene, anisole and the three xylene isomers were screened because they are analogous to toluene. PX-866 crystallized from anisole and o-xylene in a similar way to toluene. The crystallizations from toluene, anisole, and o-xylene were scaled up to 50 mg (from 10 mg) using larger solvent volumes (20 vol. equivalents) and seeding with crystals from the previous experiment instead of glass powder. The resulting solids were all crystalline by XRPD.

Residual solvents in the three solids were determined by 1H NMR integration. In each case no heptane was present but the aromatic solvent remained in roughly a 1:1 molar ratio. The material (from anisole) was dried at 90° C. under vacuum for 18 hours, which failed to remove any of the anisole. Drying under the same conditions for 88 hours removed a significant amount of the anisole (qualitatively, by HPLC), but also caused the material to turn into a glass and decompose to 95% purity (from 99%). These data (along with early stability results) led to the consensus that the crystalline forms were solvates, and PX-866 was stabilized against decomposition by interactions with solvent molecules within the crystal.

A 10 g synthesis of PX-866 was started to investigate how to incorporate the crystallization into the supplied process. Once conversion was complete by HPLC, several aliquots of the reaction mixture were removed to test various workup conditions (see experiment 16 in Table 4). In the best procedure (highest yield and purity), the mixture was dried to a thick oil, dissolved in 3.5 parts of THF, and the antisolvent (anisole/toluene) was slowly added up to 8 parts total. Due to the toxicity concern, it was opted to work up the batch with anisole and 7.9 g of PX-866 anisole solvate was isolated (78% yield). This material was 99.2% pure by HPLC (254 nm AUC), crystalline by XRPD, and contained 1.18 mol equivalents of anisole but no heptane or diallylamine.

A serial recrystallization of the anisole solvate was attempted to determine if subsequent recrystallization offered any improvement in purity (experiment 18 in Table 4). The experiment failed to yield material of >99.2% purity, and nearly 50% of the material was lost in each recrystallization. Vapor diffusion with several solvents was also attempted (experiment 19 in Table 4) in order to obtain an anhydrous, crystalline form of PX-866, but this was abandoned after one month with no positive results observed.

### Example 3

### Solubility Screening Test II

A new set of solvents (Table 2) were screened in an attempt to find a crystallization solvent that did not produce a solvate. It was found that several of the new solvents caused the compound to spontaneously crystallize immediately after dissolution. The samples that crystallized were cooled to 0° C. and subsequently −20° C. to acquire two additional temperature points. Solubility in the aliphatic solvents (propyl acetate, 4-methyl-2-pentanone, and 1-pentanol) decreased drastically with temperature from RT to −20° C.

TABLE 2
PX-866 Solubility Screening II

| | b.p. | Solubility | | |
|---|---|---|---|---|
| Solvent | (° C.) | (mg/mL) | | Comments |
| butyl acetate | 126 | >500 @ RT | | Highly soluble |
| ethyl formate | 54 | >500 @ RT | | Highly soluble |
| isopentanol | 131 | RT | 228.2 | Oiled out of 2 parts |
| cumene | 152 | RT | 4.9 | Slightly soluble, crystallized |
| | | | | from saturated solution |
| methyl acetate | 57 | >500 @ RT | | Highly soluble |
| 4-methyl-2-pentanone | 117 | RT | 38.4 | Partially soluble, crystallized |
| | | 0° C. | 33.4 | from saturated solution |
| | | −20° C. | 8.5 | |
| 1-pentanol | 138 | RT | 84.4 | Partially soluble, crystallized |
| | | 0° C. | 22.8 | from saturated solution |
| | | −20° C. | 2.4 | |
| propyl acetate | 102 | RT | 84.6 | Partially soluble, crystallized |
| | | 0° C. | 54.8 | from saturated solution |
| | | −20° C. | 18.6 | |
| chlorobenzene | 132 | RT | 15.6 | Slightly soluble, crystallized |
| | | | | from saturated solution |
| ethylene glycol | 197 | RT | 135.9 | Partially soluble |
| 2-ethoxyethanol | 135 | >500 @ RT | | Highly soluble |
| nitromethane | 101 | >500 @ RT | | Highly soluble |
| benzyl alcohol | 205 | RT | 498.0 | Highly soluble, crystallized |
| | | | | from saturated solution |
| di(ethylene glycol) | 197 | RT | 374.8 | Partially soluble |
| diethylamine | 56 | >500 @ RT | | Highly soluble |
| ethanolamine | 171 | >500 @ RT | | Solution turned dark orange, |
| | | | | no PX-866 detected in |
| | | | | supernatant |
| glycerol | 182 | RT | 12.6 | Slightly soluble |
| 1,1,1,3,3,3,hexafluoro-2- | 59 | >500 @ RT | | Highly soluble |
| propanol | | | | |
| 1-octanol | 195 | RT | 90.6 | Partially soluble |
| perfluorohexane | 59 | RT | 0.28 | Effectively insoluble |

| | | | |
|---|---|---|---|
| 1,1,2,2-tetrachloroethane | 131 | >500 @ RT | Highly soluble |
| 1,1,1-trichloroethane | 74 | >500 @ RT | Highly soluble |
| 2,2,2-trifluoroethanol | 74 | >500 @ RT | Highly soluble |

### Example 4

### Crystallization Test II

The anisole solvated PX-866 was recrystallized from propyl acetate, 4-methyl-2-pentanone, 1-pentanol, cumene, and chlorobenzene by dissolving the solid in a minimal amount of solvent at RT and cooling to −20° C. overnight. Much slower crystallization and larger crystal size resulted in the case of the aliphatic solvents. NMR residual solvent analysis was performed on each of the solids; each contained both the crystallization solvent and anisole which added to roughly 1 mol equivalent. The XRPD patterns of the solids were all remarkably similar to each other and to previous lots.

The crystallizations were repeated starting with amorphous PX-866 to see if completely excluding anisole would yield an anhydrous crystal form. However, the XRPD pattern of these crystals was indicative of the same crystal form.

The new aliphatic solvents were incorporated into the manufacturing procedure on a 10 g batch. Three aliquots of the completed reaction mixture were removed to test propyl acetate, 4-methyl-2-pentanone, and 1-pentanol in the workup (see experiment 26 in table 4). All three options resulted in acceptable yields of similarly high purity material with ˜0.8 mol equivalents of solvent. It was elected to work up the batch with propyl acetate and 8.79 g of PX-866 propyl acetate solvate was obtained (80.7% yield). The material was 99.3% pure by HPLC (254 nm AUC) and contained 0.85 mol equivalents of propyl acetate by NMR.

The DSC and TGA profiles of the anisole and propyl acetate solvates were acquired for the purpose of determining the temperature of solvent volatilization. In both cases only one DSC endotherm was observed due to melting, but the melting points of the two solvates were different (146° C. for anisole, 80.5° C. for PrOAc). The TGA results were inconclusive, as weight losses occurred over a broad temperature range and did not correspond to the solvent content. TG-IR would be a useful technique to gain insight into the solvent loss as well as decomposition pathway.

### Example 5

### Crystallization Study in Single Solvent Anisole (18 Volumes, No Glass Powder)

53 mg of PX-866 was placed in a 2 mL flat bottom vial and then added ˜940 μL (18 vol.) of anisole at RT. The supernatant was transferred to another vial and cooled down first to 4° C., and then to −20° C. (PX-866 quantified in aliquots of the supernatant at both temperatures). The resulted solids were dried under a stream of nitrogen and further dried under vacuum at RT for 16 hours, then dried under vacuum at 40° C. for 16 hours. PX-866 concentration in supernatant: RT: 9.9 mg/mL; 4° C.: 7.0 mg/mL; −20° C.: 6.2 mg/mL.

Isolated solids appear to be crystalline under microscope. The crystalline contained 15.8% (w/w) anisole, 0.8% heptane (by ¹H NMR integration) after nitrogen drying. After RT vacuum drying, no more heptane was detected and anisole amount was unchanged. After 40° C. vacuum drying the crystalline was 98.5% pure by HPLC (254 nm AUC), and anisole amount appeared unchanged by NMR. XRPD pattern of the isolated crystalline shows a crystalline material (see FIG. 1).

XRPD data: (2-theta) 7.9, 8.5, 10.2, 11.1, 14.0, 14.2, 17.9, 18.7, 21.0, 21.2, and 28.2±0.1 (See FIG. 1).

DSC data: a predominant endotherm at 146° C. (See FIG. 2).

The a-axis projection of the crystal packing in an anisole solvate of PX-866 is shown in FIG. 3. The crystalline form exhibits a single crystal X-ray crystallographic analysis at 120 K with crystal parameters as the following:

| Space Group | P2₁2₁2₁ |
|---|---|
| a, Å | 13.7140(3)° |
| b, Å | 15.4272(4) |
| c, Å | 15.6890(4) |
| α | 90 |
| β | 90 |
| γ | 90 |
| Z (molecules/unit cell) | 4° |
| Calculated Density (g/cm) | 1.268.° |

### Example 6

### Anisole Crystallization Study with PX-866 in THF Solution

To a 100 μL V-shaped glass vial containing 10 mg PX-866 was added 20 μL (2 vol.) THF for complete solubilization. Anisole (1 to 3 volumes) was slowly added to the resulting solution; solids were visible after 2 volumes of anisole. Solids formed and dried under nitrogen. Isolated solids appeared crystalline under the microscope.

A larger scale was also conducted (51 mg in 102 uL of THF and 408 uL of anisole). XRPD pattern suggests same crystal as Example 5. NMR integration shows 1.74 equivalent anisole in the crystal.

### Example 7

### Crystallization Study in Single Solvent: Toluene (2 to 5 Volumes)

About 10 mg of PX-866 was dissolved in toluene (20 μL; 2 volumes) at RT with or without glass powder added. Upon gentle agitation to dissolve solid, product crystallized on walls of vial. A larger scale experiment was also conducted. About 50 mg of PX-866 was placed in 2 mL flat bottom HPLC vial. About 250 μL (5 volumes) toluene was added to the vial at RT. The supernatant was transferred to another vial containing seeds from the 10 mg scale and cooled down first to 4° C., and then −20° C. (PX-866 quantified in aliquots of the supernatant at both temperatures). The crystalline was formed and dried under a stream of nitrogen. XRPD pattern of the isolated crystalline typical of a crystalline material.

### Example 8

### Crystallization Study in Single Solvent: Toluene (20 Volumes, No Glass Powder)

About 47 mg of PX-866 was placed in 2 mL flat bottom HPLC vial. About 1000 μL (20 volumes) toluene was added to the vial at RT. The supernatant was transferred to another vial and cooled down first to 4° C., and then −20° C. (PX-866 quantified in aliquots of the supernatant at both temperatures). The crystalline was formed and dried under a stream of nitrogen.

The crystalline contained 12.5% (w/w) toluene, 0.98% heptane (by ¹H NMR integration) after nitrogen drying. After RT vacuum drying, no more heptane was detected and toluene amount was unchanged. After 40° C. vacuum drying the crystalline was 97.7% pure by HPLC (254 nm AUC), and toluene amount appeared unchanged by NMR.

XRPD data: (2-theta) 7.9, 8.5, 10.2, 11.1, 12.5, 14.0, 18.7, and 21.1, ±0.1 (FIG. 4).

DSC data: a predominant endotherm at 142.0° C. (FIG. 5).

### Example 9

### Toluene Crystallization Study with PX-866 in THF Solution

To a 100 μL V-shaped glass vial containing 10 mg PX-866 was added 20 μL (2 vol.) THF for complete solubilization. Toluene (1 to 3 volumes) was slowly added to the resulting solution; solids were visible after 2 volumes of toluene. Evaporation of ˜50% of the initial volume under a stream of nitrogen gave a supernatant that was transferred to another vial. Solids formed and dried under nitrogen. Isolated solids appeared crystalline under the microscope.

A larger scale was also conducted (64.5 mg in 129 uL of THF and 516 uL of toluene). XRPD pattern suggests same crystal as Example 8. NMR integration shows 1.54 equivalent toluene in the crystal.

### Example 10

### Propyl Acetate Crystallization

To a 100 mg of anisole solvate in scintillation vial was added 12 volumes of propyl acetate for complete dissolution. The resulting solution was held at room temperature for 2 hours and then cooled to −20° C. overnight. The produced solids were filtered and washed with minimal amount of cold propyl acetate and dried at room temperature under vacuum overnight. The crystal was analyzed by NMR for solvent content and by XRPD for crystallinity (see FIG. 6). It was found that the crystal contains 0.76 mol equiv PrOAc and 0.34 mol equiv anisole.

XRPD data: (2-theta) 8.0, 8.4, 10.2, 11.0, 14.0 and 19.2, ±0.1. (FIG. 6)

DSC data: a predominant endotherm at 80.5° C. (FIG. 7)

The a-axis projection of the crystal packing in a propyl acetate solvate of PX-866 is illustrated in FIG. 8. The crystalline propyl acetate solvate exhibits a single crystal X-ray crystallographic analysis at 100 K with crystal parameters as the following:

| Space Group | P2₁2₁2₁ |
|---|---|
| a, Å | 13.4963(5)° |
| b, Å | 15.5158(5) |
| c, Å | 15.6912(6) |
| α | 90 |
| β | 90 |
| γ | 90 |
| Z (molecules/unit cell) | 4° |
| Calculated Density (g/cm) | 1.269.° |

### Example 11

### 4-Methyl-2-pentanone crystallization

To a 100 mg of anisole solvate in scintillation vial was added 25 volumes of 4-methyl-2-pentanone for complete dissolution. The resulting solution was held at room temperature for 2 hours and then cooled to −20° C. overnight. The produced solids were filtered and washed with minimal amount of cold 4-methyl-2-pentanone and dried at room temperature under vacuum overnight. The crystal was analyzed by NMR for solvent content and by XRPD for crystallinity. It was found that the crystal contains 0.90 mol equiv 4-methyl-2-pentanone and 0.15 mol equiv anisole.

XRPD data: (2-theta) 7.9, 8.4, 10.2, 10.9, 13.9, 14.2, 18.5, 19.2, and 20.7, ±0.1. (FIG. 9)

### Example 12

### Cumene Crystallization

To a 100 mg of anisole solvate in scintillation vial was added 221 volumes of cumene for complete dissolution. The resulting solution was held at room temperature for 2 hours and then cooled to −20° C. overnight. The produced solids were filtered and washed with minimal amount of cold cumene and dried at room temperature under vacuum overnight. The crystal was analyzed by NMR for solvent content and by XRPD for crystallinity. It was found that the crystal contains 0.96 mol equiv cumene and 0.06 mol equiv anisole.

XRPD data: (2-theta) 7.8, 8.4, 10.1, 10.7, 13.7, 14.1, 18.1, 18.9, 20.6, and 20.8, ±0.1. (See FIG. 10).

### Example 13

### 1-Pentanol Crystallization

To a 100 mg of anisole solvate in scintillation vial was added 35 volumes of 1-pentanol for complete dissolution. The resulting solution was held at room temperature for 2 hours and then cooled to −20° C. overnight. The produced solids were filtered and washed with minimal amount of cold 1-pentanol and dried at room temperature under vacuum overnight. The crystal was analyzed by NMR for solvent content and by XRPD for crystallinity. It was found that the crystal contains 0.46 mol equiv 1-pentanol and 0.77 mol equiv anisole.

XRPD data: (2-theta) 8.1, 8.5, 10.2, 11.1, 12.5, 14.0, 14.3, 17.9, 18.8, 20.7, and 21.3, ±0.1. (FIG. 11)

### Example 14

### Chlorobenzene Crystallization

To a 100 mg of anisole solvate in scintillation vial was added 52 volumes of chlorobenzene for complete dissolution. The resulting solution was held at room temperature for 2 hours and then cooled to −20° C. overnight. The produced solids were filtered and washed with minimal amount of cold chlorobenzene and dried at room temperature under vacuum overnight. The crystal was analyzed by NMR for solvent content and by XRPD for crystallinity. It was found that the crystal contains 0.86 mol equiv chlorobenzene and 0.15 mol equiv anisole.

XRPD data: (2-theta) 8.0, 8.5, 10.3, 11.1, 14.1, 17.9, 18.8, 19.1, 21.0 and 28.3, ±0.1 (FIG. 12).

### Example 15

### Synthesis of PX-866 Anisole Solvate from Wortmannin

In a 250 mL RBF equipped with a nitrogen bubbler, thermocouple, magnetic stirring, and cooling water bath, wortmannin (10 g, 23.34 mmol) was suspended in 50 mL of anhydrous THF resulting in a thin yellow slurry. To the slurry, diallylamine (34.5 mL, 280.1 mmol) was slowly added maintaining the temperature below 30° C. A 10 minute COR sample showed ˜2% wortmannin remaining. After 90 minutes no wortmannin was detected. Six 2.5 mL aliquots of the reaction mixture were removed to test workup conditions; meanwhile the remaining solution was stored at −20° C. The solvent was removed to afford a thick orange oil and THF (29 mL, 3.5 vol) was added and stirred until homogeneous. Anisole (25 mL, 3 vol) was added and the solution was seeded with anisole solvate crystals. Slowly, additional anisole was added up to a total of 8 vol. The suspension was agitated overnight. The solid was isolated on a Buchner funnel and washed with 2 vol of 3.5:8 THF/anisole. The isolated solid was dried overnight at RT in a vacuum oven to afford 7.9 g of PX-866 anisole solvate (78% yield).

### Example 16

### Synthesis of PX-866 Propyl Acetate Solvate from Wortmannin

In a 250 mL RBF equipped with a nitrogen bubbler, thermocouple, magnetic stirring, and cooling water bath, wortmannin (10 g, 23.34 mmol) was suspended in 50 mL of anhydrous THF resulting in a thin yellow slurry. To the slurry, diallylamine (3.5 mL, 28.0 mmol) was slowly added maintaining the temperature below 30° C. A 60 minute COR sample showed 0.2% wortmannin remaining. After 90 minutes, three 2 mL aliquots of the reaction mixture were removed to investigate workup conditions; meanwhile the remaining solution was stored at −20° C. The solvent was removed to afford a thick orange oil and propyl acetate (45 mL, 5 vol) was added and stirred until homogeneous. After ˜1 minute the solution became cloudy. Agitation was continued at RT for 2 hours, and then the solution was cooled to −20° C. overnight. The solid was isolated on a Buchner funnel and washed with 1 vol. of cold propyl acetate. The isolated solid was dried on a rotovap overnight (30° C. bath temp, 8 mm Hg) to afford 8.79 g of PX-866 propyl acetate solvate (80.7% yield).

### Example 17

### Stability Test

A non-GMP stability study was initiated to establish the stability of the PX-866 solvates against heat and humidity compared to that of the amorphous material. Samples of the anisole solvate, toluene solvate, and amorphous PX-866 were placed in a stability chamber set to 40° C. and 75% relative humidity to be pulled at 1, 2, 3, 4, and 8 week time points for HPLC analysis. As soon as it was available, the propyl acetate solvate was added to the chamber to be pulled at a single 2.5 week point. (See FIG. 13).

The anisole and toluene solvates offered a clear upgrade in stability over amorphous PX-866, which was evident after only one week. Based on limited data (only a single time point), the propyl acetate solvate was more stable than the amorphous material but less stable than the anisole solvate.

After the 8 week period in the stability chamber, the materials were analyzed by NMR for solvent content and XRPD for crystallinity. The anisole and toluene solvates contained 1.34 and 1.25 mol equivalents of solvent respectively, which is roughly comparable to the original amount. The propyl acetate solvate contained 0.36 mol equivalents of solvent (roughly half the original amount) as well as a significant amount of water. All three solids were crystalline by XRPD.

### Example 18

### Amorphous and Crystalline PX-866 API-In-Capsule in Rat Oral Pharmacokinetic Study

Absorption of orally administered amorphous and crystalline anisole solvate of PX-866 in rats (fed or fast) has been studied to investigate their pharmacokinetics in vivo. Rats were given 0.3 mg of amorphous and crystalline anisole solvate of PX-866, and blood was collected after administration.

| | Dose | | | |
|---|---|---|---|---|
| Test Article | (mg) | Food | Species | Formulation |
| Amorphous PX-866 | 0.3 | Fast | SD rat | API-in-capsule |
| Crystalline PX-866 | 0.3 | Fast | SD rat | API-in-capsule |
| Amorphous PX-866 | 0.3 | Fed | SD rat | API-in-capsule |
| Crystalline PX-866 | 0.3 | Fed | SD rat | API-in-capsule |

### Results

There is no statistically significant difference between plasma concentrations of PX-866 or the metabolite 17-OH-PX-866 in fed or fasted rats after administration of amorphous powder in capsules or crystalline anisole solvate PX-866 powder in capsules.

### Example 19

### Crystalline/Solvate PX-866 Formulations

### Example 19a

### Hard Gelatin Capsule for Oral Administration

To prepare a pharmaceutical formulation for oral administration, 10 mg of crystalline anisole solvate of PX-866 in powder form was directly filled into hard gelatin capsules with no processing or blending with any excipients. The capsules were individually opened, tared, filled and closed with a capsule filling machine (Xcelodose, Capsugel).

### Example 19b

### Direct-Compression Tablet for Oral Administration

To prepare a pharmaceutical formulation for oral administration, crystalline anisole solvate of PX-866, Mg-Stearate, and Mannitol (Pearlitol 100SD, Roquette) were weighed, sieved and subsequently dry-blended (Turbula mixer). The API and mannitol were blended first and the dry blend is tested for uniformity. After passing that test, the Mg-Stearate was added for a short additional blend and the material was then tableted by a direct compression tabletting machine with appropriate weight and hardness checks. 10 mg tablets of crystalline PX-866 were produced.

Tablets were then spray coated with a yellow moisture barrier (Opadry AMB 80W120002 Yellow) to both protect patients and pharmacists from directly contacting the API as well as to protect the API from possible moisture ingress.

### Example 19c

### Hard Lozenge for Buccal Administration

To prepare a sublingual pharmaceutical formulation for buccal delivery, e.g., hard lozenge, 10 mg of crystalline anisole solvate of PX-866 is mixed with 490 mg of powdered sugar, 1.6 ml of light corn syrup, 2.4 mL distilled water and 0.42 mL mint extract. The mixture is gently blended and poured into mold to form a lozenge suitable for buccal administration.

### Example 19d

### Injectable Solution for Parenteral Administration

To prepare a parenteral pharmaceutical formulation suitable for administration by injection, 10 mg of an anisole solvate of PX-866 is mixed with 10 mL of 0.9% sterile saline. The mixture is incorporated into a dosage unit form suitable for administration by injection.

### Example 19e

### PX-866 Anisole Solvate 2 mg Tablets

Large prismatic crystals of the anisole solvate (see FIG. 17(A), FIG. 17(B)) are reduced to a uniform size by sieving prior to charging a blending vessel with the PX-866 anisole solvate.

Pearlitol 100SD, a size controlled granulated crystalline form of mannitol is used. The mean diameter of the particles is 100 microns. The Pearlitol is sieved prior to blending to ensure no agglomeration is present.

Magnesium stearate is sieved prior to blending to eliminate any agglomerates. The PX-866 anisole solvate and excipients were charged to a Turbula® blender and blended for 19 minutes. An additional charge of magnesium stearate (for a total of 2% of the batch weight) was added to the bulk dry blend and the blending was continued for an additional 2-4 minutes. The powder was visually homogenous with no stratification. The blend was compressed to tablets. The tablets were then coated with Yellow Opadry® AMB in a pan coater. The table below shows a batch formula for 25,000 tablets of PX-866 anisole solvate, 2 mg.

| | | | Theoretical Quantity |
|---|---|---|---|
| Material Name | | Quantity per | Required for 25,000 |
| (Chemical) | Grade | Unit (mg) | Units (grams) |
| PX-866 Anisole | cGMP | 2.5 | 62.5 |
| Solvate | | | |
| Pearlitol ® 100SD | USP | 146.0 | 3,650.0 |
| Magnesium Stearate | NF | 3.0 | 75.0 |
| Opadry ® AMB, | GRAS | 6.1 | 152.5 |
| Yellow | | | |

| | | |
|---|---|---|
| Total | 157.6 | 3,940.0 |

The table below shows the amounts of excipient per 2 mg tablet. Tablets are coated to a bulk weight gain of no less than 4%. For a 150 mg average tablet weight, the dried coating weight per tablet is about 6 mg.

| Excipient | Chemical | Amount per tablet | |
|---|---|---|---|
| Pearlitol ® 100 SD | mannitol | 146 | mg |
| (100 micron granulated) | | | |
| Magnesium stearate | Mg stearate | 3.0 | mg |
| Yellow Opadry ® AMB | polymer | 6 | mg |

### Example 20

### Clinical Trial

A Phase 1 two-way cross-over study of the pharmacokinetics and pharmacodynamics of crystalline PX-866 tablets and amorphous PX-866 capsules administered in the fasting state, and of crystalline PX-866 tablets administered fed and fasting, in healthy subjects

### Study Objectives:

### Primary:

Part 1: To evaluate and compare the pharmacokinetic (PK) profiles (of PX-866 and metabolites) after administration of crystalline PX-866 tablets and amorphous PX-866 capsules.

Part 2: To evaluate the effect of food on the PK profile of crystalline PX-866 tablets.

### Secondary:

To evaluate the safety and tolerability of crystalline PX-866 tablets.

### Exploratory:

To explore the pharmacodynamic effects of PX-866 on the activation status of proteins in the PI-3K pathway in platelets and on the fasting levels of plasma C-peptide.

### Study Population

Healthy subjects between ages 18 and 65.

### Study Design

This is a two part, Phase 1, open label, cross over study designed to evaluate the PK profile of crystalline PX-866 tablets relative to that of the amorphous PX-866 capsules and to evaluate the effect of administration with food on the PK of crystalline PX-866.

Each enrolled subject will participate in only one Part of the study. Subjects in part 1 of the study will receive two single dose 8 mg treatments of PX-866 (one each of crystalline PX-866 tablets and amorphous PX-866 capsules) in Periods A and B, separated by at least seven days. Subjects in part 2 of the study will receive two single dose 6 mg treatments of PX-866 (crystalline PX-866 tablets administered in either fed or fasted state), in Periods C and D, separated by at least seven days.

Each subject will be scheduled to return for a final visit approximately one week after the last administration of study drug.

### Test Product, Dose, and Mode of Administration

Part 1:

Amorphous PX-866 capsules or crystalline PX-866 tablets administered orally at 8 mg on Day 1 and Day 8.

Part 2:

Crystalline PX-866 tablets administered orally at 6 mg on Day 1 and Day 8 in a fasting or fed state.

### Number of Planned Subjects

Enrollment of up to approximately 46 subjects, such that approximately 36 evaluable subjects (24 subjects in part 1 and 12 subjects in part 2) will be evaluable.

### Duration of Treatment

Two weeks. Each subject will be scheduled to return for safety follow-up 7 days after the last dose of PX-866.

### Safety Assessments

Safety assessments will include documentation of adverse events, ECG and laboratory results.

### Pharmacokinetic Assessments

Pharmacokinetic assessments will include measurement of plasma levels of PX-866 and metabolites.

### Pharmacodynamic Assessments

Pharmacodynamic assessments, to be performed in Part 1 only, will include evaluation of changes in fasting C-peptide and phosphorylation status of PI-3K pathway signaling proteins including but not limited to AKT, EGFR, mTOR, and S6 in platelets.

### Pharmacokinetic Methods

Pharmacokinetic parameters for PX-866 and relevant metabolites will be derived from plasma concentration versus time data using standard non-compartmental methods. The pharmacokinetic parameters to be assessed include, but are not necessarily limited to:

Cₘₐₓ: Maximum observed plasma concentration

Tₘₐₓ: Time from time zero (time of dose administration) of maximum observed plasma concentration AUCₗₐₛₜ: Area under the plasma concentration-time curve from time zero to the last quantifiable time point AUC∞: Area under the plasma concentration-time curve from time zero extrapolated to infinity t_{1/2}: Terminal half-life

Actual collection times will be used for the analysis. Additional details on the pharmacokinetic methods and analysis will be provided in the statistical analysis plan (SAP).

### Statistical Methods

### Treatment Assignment

In each part of the study, subjects will be assigned to one of two alternating dosing sequences for the two treatments to be administered: Crystalline PX-866 tablets and amorphous PX-866 capsules (part 1); crystalline PX-866 tablets fed and fasted (part 2). The sequence of the treatments will be assigned in accordance with the subject's study number (odd or even) assigned at the time of registration. No stratification factors will be used. No blinding will be used.

### Sample Size Determination

Part 1:

This is a pilot evaluation of the pharmacokinetics of two oral drug formats of PX-866, and as such, is not formally designed to demonstrate bioequivalence. However, assuming reasonable intersubject and intrasubject variability will be observed in part 1, a sample size of approximately 24 evaluable subjects (12 per group) is anticipated to provide sufficient estimates of PX-866 pharmacokinetic parameters for the two drug formats to inform dose administration guidelines for the crystalline PX-866 tablets in future PX-866 clinical studies. In addition, a sample size of 24 would be sufficient to demonstrate bioequivalence (assuming no sequence effect and that equivalence defined as the ratio for means should be within 80-125%) with a one-sided alpha of 0.05 and 80% power if the true intersubject CV is 75% and intrasubject CV is 20% for AUC.

Part 2:

No formal hypothesis is to be tested, and therefore no formal sample size calculations were performed. However, a sample size of 12 subjects should provide a preliminary estimate of the effect of food on the pharmacokinetic parameters of crystalline PX-866 tablets.

### Statistical Analysis

Adjusted means will be produced for all variables. For transformed variables, adjusted means on the log scale will be back-transformed to provide geometric means on the original scale of measurement. Treatment (crystalline vs. amorphous or fed vs. fasted) contrasts will be estimated and 90% confidence intervals generated. These contrasts will be based on differences for untransformed variables and ratios for the log-transformed variables, using amorphous PX-866 capsule values (part 1) or fasted crystalline PX-866 tablet values (part 2) as reference.

To assess the effect of the two drug formulations or fed vs. fasting state on oral bioavailability, analysis of variance (ANOVA) will be performed on log-transformed AUC ∞ and C max, values. Factors in the ANOVA will include sequence, subject within sequence, period and treatment. Point estimates and 90% confidence intervals for means and differences between means on the log scale will be exponentiated to obtain estimates and ratios of geometric means on the original scale. The 90% confidence interval for the ratio of population geometric means of crystalline tablet to amorphous capsule treatments (part 1) or fed vs. fasted state (part 2) will be evaluated to ascertain effects of formulation or food on pharmacokinetics of PX-866. Summary statistics will be tabulated by treatment (crystalline and amorphous; fed vs. fasted) for all pharmacokinetic parameters. Individual pharmacokinetic parameters will be listed for each subject. Adjusted means will be produced for all variables. For transformed variables, adjusted means on the log scale will be back-transformed to provide geometric means on the original scale of measurement. Treatment contrasts will be estimated and 90% confidence intervals generated. These contrasts will be based on differences for untransformed variables and ratios for the log-transformed variables, using amorphous values (part 1) or fasted values (part 2) as reference.

### Primary Objective

Part 1: To evaluate and compare the pharmacokinetic (PK) profiles (of PX-866 and metabolites) after administration of crystalline PX-866 tablets and amorphous PX-866 capsules.

Part 2: To evaluate the effect of food on the PK profile of crystalline PX-866 tablets.

### Secondary Objectives

To evaluate the safety and tolerability of crystalline PX-866 tablets.

### Exploratory Objectives

To explore the pharmacodynamic effects of PX-866 on the activation status of proteins in the PI-3K pathway in platelets and on the fasting levels of plasma C-peptide.

### Endpoints

### Primary Endpoint

Cₘₐₓ, Tₘₐₓ, AUCₗₐₛₜ, AUC∞ and t_{1/2} of plasma concentrations of PX-866 and PX-866 metabolites

### Secondary Endpoints

Incidence and severity of adverse events, vital sign, clinical laboratory, and ECG changes or abnormalities

### Exploratory Endpoints

Changes in phosphorylation status of PI-3K pathway signaling proteins (including but not limited to AKT, EGFR, mTOR, S6) in platelets as well as changes in fasting C-peptide levels.

### Example 21

### Non-Invasive Pharmacodynamic Assay

In order to non-invasively monitor clinical drug pharmacodynamics, an assay was developed which utilizes platelets to quantify PI-3K pathway inhibition following exposure to PX-866. In this assay, isolated platelets are stimulated for 15 minutes at 37° C. using thrombin activating peptide (TRAP) to activate PI-3K signaling. After stimulation, lysates are prepared from the platelets and analysed for both phosphorylated Akt (P-Akt) and total Akt (T-Akt) levels using commercially available human ELISA kits. Analysis of the in vitro dose response relationship of PX-866 in this assay, using platelets isolated from three different donors, is shown in FIG. 14. For comparative purposes the in vitro dose response relationship of PX-866 using two different human tumor-derived cell lines (MCF-7 and A549) are included in the figure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A crystalline form of a compound having a structural formula [chemical structure]
which is substantially free of wortmannin.

2. A crystalline form of a compound having a structural formula [chemical structure]
wherein the form is
(a) a crystalline anisole solvate; and
(b) has an X-ray powder diffraction pattern (XRPD) with characteristic peaks at 7.9±0.1 degrees 2-Theta, 8.5±0.1 degrees 2-Theta, 10.2±0.1 degrees 2-Theta, 11.1±0.1 degrees 2-Theta, 14.0±0.1 degrees 2-Theta, 14.2±0.1 degrees 2-Theta, 17.9±0.1 degrees 2-Theta, 18.7±0.1 degrees 2-Theta, 21.0±0.1 degrees 2-Theta, 21.2±0.1 degrees 2-Theta, and 28.2±0.1 degrees 2-Theta.

3. (canceled)

4. The crystalline form of claim 3, having a purity of at least 90%.

5. The crystalline form of claim 3, having a purity of at least 95%.

6. The crystalline form of claim 2, wherein the crystalline form exhibits a predominant endotherm at about 146° C. as measured by Differential Scanning calorimeter.

7. The crystalline form of claim 2, wherein the form has the general space group P2₁2₁2₁.

8. The crystalline form of claim 2, wherein the crystalline form exhibits a single crystal X-ray crystallographic analysis at 120 K with the following crystal parameters:

| Space Group | P2₁2₁2₁ |
|---|---|
| a, Å | 13.7140(3)° |
| b, Å | 15.4272(4) |
| c, Å | 15.6890(4) |
| α | 90 |
| β | 90 |
| γ | 90 |
| Z (molecules/unit cell) | 4° |
| Calculated Density (g/cm) | 1.268.° |

9. (canceled)

10. A method of preparing a crystalline solvate form of a compound having a structural formula [chemical structure]
that is substantially free of wortmannin, comprising cooling down a supernatant, solution, suspension, dispersion or emulsion of the compound in a suitable solvent to a temperature of between 4° C. to −20° C.

11. The method of claim 10 wherein the supernatant, solution, suspension, dispersion or emulsion comprises a solvent selected from toluene, anisole, cumene, propyl acetate, 4-methyl-2-pentanone, chlorobenzene, or 1-pentanol, or a combination thereof.

12. The method of claim 10 wherein the supernatant, solution, suspension, dispersion or emulsion comprises anisole.

13. The method of claim 10 wherein the method comprises adding an antisolvent to the supernatant, solution, suspension, dispersion or emulsion of the compound in the solvent, wherein Compound 1 has differential solubility in the solvent compared to the antisolvent.

14. The method of claim 13, comprising optionally cooling the supernatant, solution, suspension, dispersion or emulsion of the compound in the solvent to a temperature of between 4° C. to −20° C. prior to adding an antisolvent.

15. The method of claim 13 wherein the solvent is selected from tetrahydrofuran (THF), water, acetonitrile, acetone, n-butanol, sec-butanol, butyl acetate, tert-butylmethyl ether (TBME), chloroform, 1,2-dichloroethane, N,N-dimethylacetamide, N,N-dimethylformamide, dimethyl sulfoxide, ethanol, 1,4-dioxane, ethyl acetate, isopropyl acetate, isobutyl acetate, 2-ethoxyethanol, ethylene glycol, formamide, methanol, 2-methoxyethanol, methylbutyl ketone, N-methylpyrrolidone, nitromethane, pyridine, sulfolane, toluene, xylene, anisole, hexane, cyclohexane, methylcyclohexane, cumene, propyl acetate, chlorobenzene, pentane, 1-pentanol, 4-methyl-2-pentanone and 1,1,2-trichloroethene, or a combination thereof.

16. The method of claim 13 wherein the antisolvent is selected from water, toluene, anisole, cumene, propyl acetate, 4-methyl-2-pentanone, chlorobenzene, or 1-pentanol, or a combination thereof.

17. The crystalline form of claim 2 wherein the form has an XRPD of FIG. 1.

18. (canceled)

19. (canceled)

20. (canceled)

21. (canceled)

22. (canceled)

23. (canceled)

24. (canceled)

25. A pharmaceutical composition comprising the crystalline form of claim 2, and a pharmaceutically acceptable carrier.

26. The pharmaceutical composition of claim 25, wherein the form is present in a unit dosage form in an amount of about 0.1 to 20 mg.

27. The pharmaceutical composition of claim 25, wherein the pharmaceutical composition further comprises a second anti-cancer agent.

28. (canceled)

29. (canceled)

30. A method of treating cancer in a subject in need thereof comprising administering a composition comprising a therapeutically effective amount of the crystalline form of claim 2 to the subject in need thereof.

31. (canceled)

32. (canceled)

33. The method of 30, wherein the method further comprises administering an anti-cancer agent.

34. (canceled)
